(19) United States
(12) Patent Application Publication
(10) Pub. No.: US 2026/0263042 A1
(43) Pub. Date: Sep. 10, 2026

(54) VOICE ACTIVATED COMMANDS FOR OPERATING IMAGING SYSTEMS

(71) Applicant: Boston Scientific Scimed, Inc., Maple Grove, MN (US)

(72) Inventors: Alan Gerard Torborg, Minneapolis, MN (US); Zhichao Hong, Maple Grove, MN (US); Yuan Ren, Maple Grove, MN (US)

(73) Assignee: Boston Scientific Scimed, Inc., Maple Grove, MN (US)

(21) Appl. No.: 19/559,908

(22) Filed: Mar. 6, 2026

Related U.S. Application Data
(60) Provisional application No. 63/768,402, filed on Mar. 7, 2025.

(51) Int. Cl. A61B 8/00; A61B 8/12; G10L 15/06; G10L 15/08; G10L 15/22

(52) U.S. Cl. CPC A61B 8/467; A61B 8/12; A61B 8/461; A61B 8/54; G10L 15/063; G10L 15/08; G10L 15/22; G10L 2015/088; G10L 2015/223

(57) ABSTRACT

The present disclosure provides for the use of voice activated commands to control the operation of an intravascular ultrasound (IVUS) imaging system and/or to control a graphical user interface (GUI) arranged to convey information related to the IVUS images and other data generated by and for controlling the operations performed by the IVUS imaging system.

### CROSS-REFERENCE TO RELATED APPLICATION

This application claims the benefit of U.S. Patent Application Ser. No. 63/768,402, filed Mar. 7, 2025, entitled “VOICE ACTIVATED COMMANDS FOR OPERATING IMAGING SYSTEMS”, which is incorporated by reference herein in its entirety.

### TECHNICAL FIELD

The present disclosure generally relates to intravascular ultrasound (IVUS) imaging systems. Particularly, but not exclusively, the present disclosure relates to use of voice activated commands and speech recognition for controlling operations associated with IVUS imaging systems.

### BACKGROUND

Ultrasound devices insertable into patients have proven diagnostic capabilities for a variety of diseases and disorders. For example, intravascular ultrasound (IVUS) imaging systems have been used as an imaging modality for diagnosing blocked blood vessels and providing information to aid medical practitioners in selecting and placing stents and other devices to restore or increase blood flow.

IVUS imaging systems include a control module (with a pulse generator, an image acquisition and processing components, and a monitor), a catheter, and a transducer disposed in the catheter. The transducer-containing catheter is positioned in a lumen or cavity within, or in proximity to, a region to be imaged, such as a blood vessel wall or patient tissue in proximity to a blood vessel wall. The pulse generator in the control module generates electrical pulses that are delivered to the transducer and transformed into acoustic pulses that are transmitted through patient tissue. The patient tissue (or other structure) reflects the acoustic pulses and reflected pulses are absorbed by the transducer and transformed into electric pulses. The transformed electric pulses are delivered to the image acquisition and processing components and converted into images displayable on the monitor.

### BRIEF SUMMARY

This Summary is provided to introduce a selection of concepts in a simplified form that are further described below in the Detailed Description. This Summary is not intended to necessarily identify key features or essential features of the claimed subject matter, nor is it intended as an aid in determining the scope of the claimed subject matter.

In general, the present disclosure provides an improvement to computing devices and particularly to IVUS imaging systems in that the present disclosure provides for systems, devices, methods, and techniques utilizing voice activated commands and speech recognition technology to provide inputs to and control various operations associated with procedures performed by IVUS imaging systems. The systems, devices, methods and techniques as described herein allow for the benefits of voice activated and hands-free control of medical devices, such as but not limited to IVUS imaging systems, and thereby include many advantages over systems that require physical contact to provide inputs to the devices, especially when utilizing these devices in a sterile field environment.

In some embodiments, the disclosure of the present application can be implemented as a first method for an intravascular ultrasound (IVUS) imaging system, the first method comprising: receiving, from an audio input device coupled to a computing device, an audio signal representative of an audio sound input; processing the audio signal to detect an indication of a voice activated command included in the audio sound input; and upon detection of the indication of the voice activated command, configuring one or more operating parameters of the IVUS imaging system based on a command instruction included in the voice activated command.

With some embodiments of the first method above, the first method further comprises: wherein the voice activated command comprises a keyword followed by the command instruction.

With some embodiments of the first method above, the first method further comprises: wherein processing the audio signal comprises using speech recognition software to detect the indication of a voice activated command included in the audio sound input.

With some embodiments of the first method above, the first method further comprises: creating a voice training file using machine learning to train the speech recognition software to detect and remove an accent from the audio sound input.

With some embodiments of the first method above, the first method further comprises: wherein processing the audio signal using the speech recognition software includes using an algorithm comprising the steps of: monitoring the audio signal representative of audio sound input detected at the audio input device; detecting the presence of a keyword in the audio signal; processing a subsequent portion of the audio signal to determine that the input signal includes representations of recognized command words or instructions; and generating and outputting one or more control commands, the one or more control commands configured to control one or more aspects of the operation of one or more devices included in the IVUS imaging system.

With some embodiments of the first method above, the first method further comprises: wherein the one or more control commands are configured to control the operation of an imaging catheter while the imaging catheter is operated to perform an imaging procedure while positioned in a vessel of a patient.

With some embodiments of the first method above, the first method further comprises: wherein the one or more control commands are configured to control a content and a format of a visual display being provided on a display device coupled with the IVUS imaging system.

With some embodiments of the first method above, the first method further comprises: wherein configuring the one or more operating parameters of the IVUS imaging system based on the command instruction included in the voice activated command comprises: determining that the command instruction includes a request to display of list of procedures available for performing an imaging procedure using the IVUS imaging system; generating graphical indications comprising the list of procedures that are available for performing the imagines procedure using the IVUS imaging system, and displaying, on a display device, the list of procedure.

With some embodiments of the first method above, the first method further comprises: wherein configuring one or more operating parameters of the IVUS imaging system based on a command instruction comprises: determining that the command instruction includes an indication of a selected procedure that was chosen from a list of pre-programmed procedures available for controlling an imaging procedure using the IVUS imaging system; generating graphical indications comprising a process identifier and a listing of a set of parameters associated with the selected procedure, and displaying, on a display device, the process identifier and the set of parameters associated with the selected procedure.

With some embodiments of the first method above, the first method further comprises: wherein configuring the one or more operating parameters of the IVUS imaging system based on a command instruction comprises: determining that the command instruction includes a request to create a new pre-programmed procedure available for controlling an imaging procedure using the IVUS imaging system; generating graphical indications comprising an interactive listing of a set of parameters required for creating the new pre-programmed procedure; displaying, on a display device, the interactive listing of the set of parameters required for creating the new pre-programmed procedure, wherein the interactive listing of the set of parameters is initially displayed without parameter values associated with any of the parameters of the set of parameters; and assigning a parameter value to each of the parameters included in the set of parameters using one or more voice activated commands.

With some embodiments of the first method above, the first method further comprises: wherein the audio input device comprises a microphone incorporated into a headset.

With some embodiments of the first method above, the first method further comprises: performing an imaging procedure using the IVUS imaging system, the imaging procedure performed based at least in part on the one or more operating parameters configured based on the command instruction included in the voice activated command.

With some embodiments of the first method above, the first method further comprises: wherein performing the imaging procedure includes performing the imaging procedure using an automatic pullback of the imaging catheter through a vessel of a patient, and wherein at least one of the one or more operating parameters including a value for a drag speed for the automatic pullback.

Embodiments of the present disclosure may be implemented as an apparatus, the apparatus comprising: a processor coupled to a memory, the memory comprising instructions executable by the processor, the processor configured to couple to an intravascular ultrasound (IVUS) imaging system and configured to execute the instructions, which instructions when executed cause the processor to implement any of the embodiments of the first method above.

Embodiments of the present disclosure may be implemented as a first at least one machine readable storage device, comprising: a plurality of instructions that in response to being executed by a processor of an intravascular ultrasound (IVUS) imaging system cause the processor to implement the method of any of the embodiments of the first method above.

Embodiments of the present disclosure may be implemented as an intravascular ultrasound (IVUS) imaging system, the IVUS imaging system comprising: an audio input device; a display device; an IVUS catheter; a computing device including a processor coupled to the audio input device, the display device, and the IVUS catheter; and a memory device comprising instructions, which when executed by the processor cause the IVUS imaging system to: receive from the audio input device an audio signal representative of an audio sound input; process the audio signal to detect an indication of a voice activated command included in the audio sound input; and upon detection of the indication of the voice activated command, configure one or more operating parameters of the IVUS imaging system based on a command instruction included in the voice activated command.

With some embodiments of the IVUS imaging system, the IVUS imaging system further comprises: wherein the voice activated command comprises a keyword followed by the command instruction.

With some embodiments of the IVUS imaging system, the IVUS imaging system further comprises: the memory storage device further comprising instructions executable by the processor, which instructions when executed cause the IVUS system to: process the audio signal using speech recognition software to detect the indication of a voice activated command included in the audio sound input.

With some embodiments of the IVUS imaging system, the IVUS imaging system further comprises: the memory storage device further comprising instructions executable by the processor, which instructions when executed cause the IVUS system to: create a voice training file using machine learning to train the speech recognition software to detect and remove an accent from the audio sound input.

With some embodiments of the IVUS imaging system, the IVUS imaging system further comprises: the memory storage device further comprising instructions executable by the processor, which instructions when executed cause the IVUS system to: process the audio signal using the speech recognition software includes the use of an algorithm comprising steps configured to: monitor the audio signal representative of audio sound input detected at the audio input device; detect the presence of a keyword in the audio signal; process a subsequent portion of the audio signal to determine that the input signal includes representations of recognized command words or instructions; and generate and output one or more control commands, the one or more control commands configured to control one or more aspects of the operation of one or more devices included in the IVUS imaging system.

With some embodiments of the IVUS imaging system, the IVUS imaging system further comprises: the memory storage device further comprising instructions executable by the processor, which instructions when executed cause the IVUS system to: control the operation of an imaging catheter while the imaging catheter is operated to perform an imaging procedure while positioned in a vessel of a patient.

With some embodiments of the IVUS imaging system, the IVUS imaging system further comprises: wherein the one or more control commands are configured to control a content and a format of a visual display being provided on a display device coupled with the IVUS imaging system.

With some embodiments of the IVUS imaging system, the IVUS imaging system further comprises: wherein the one or more operating parameters of the IVUS imaging system based on the command instruction included in the voice activated command comprise instructions executable by the processor, which instructions when executed cause the IVUS system to: determine that the command instruction includes a request to display of list of procedures available for performing an imaging procedure using the IVUS imaging system; generate graphical indications comprising the list of procedures that are available for performing the imagines procedure using the IVUS imaging system, and display, on a display device, the list of procedure.

With some embodiments of the IVUS imaging system, the IVUS imaging system further comprises: wherein the one or more operating parameters of the IVUS imaging system based on a command instruction included in the voice activated command comprise instructions executable by the processor, which instructions when executed cause the IVUS system to: determine that the command instruction includes an indication of a selected procedure that was chosen from a list of pre-programmed procedures available for controlling an imaging procedure using the IVUS imaging system; generate graphical indications comprising a process identifier and a listing of a set of parameters associated with the selected procedure, and display, on a display device, the process identifier and the set of parameters associated with the selected procedure.

With some embodiments of the IVUS imaging system, the IVUS imaging system further comprises: the memory storage device further comprising instructions executable by the processor, which instructions when executed cause the IVUS system to: configure the one or more operating parameters of the IVUS imaging system based on a command instruction comprising instructions to: determine that the command instruction includes a request to create a new pre-programmed procedure available for controlling an imaging procedure using the IVUS imaging system; generate graphical indications comprising an interactive listing of a set of parameters required for creating the new pre-programmed procedure; display, on a display device, the interactive listing of the set of parameters required for creating the new pre-programmed procedure, wherein the interactive listing of the set of parameters is initially displayed without parameter values associated with any of the parameters of the set of parameters; and assign a parameter value to each of the parameters included in the set of parameters using one or more voice activated commands.

With some embodiments of the IVUS imaging system, the IVUS imaging system further comprises: wherein the audio input device comprises a microphone incorporated into a headset.

With some embodiments of the IVUS imaging system, the IVUS imaging system further comprises: the memory storage device further comprising instructions executable by the processor, which instructions when executed cause the IVUS system to: perform an imaging procedure using the IVUS imaging system, the imaging procedure performed based at least in part on the one or more operating parameters configured based on the command instruction included in the voice activated command.

With some embodiments of the IVUS imaging system, the IVUS imaging system further comprises: wherein the instructions executable by the processor, which instructions when executed cause the IVUS system to perform the imaging procedure include instructions to: perform the imaging procedure using an automatic pullback of the imaging catheter through a vessel of a patient, and wherein at least one of the one or more operating parameters including a value for a drag speed for the automatic pullback.

Embodiments the present disclosure may be implemented as a second embodiment of an at least one machine readable storage device, the second embodiment of the at least one machine readable storage device comprising: a plurality of instructions that in response to being executed by a processor of an intravascular ultrasound (IVUS) imaging system cause the processor to: receive from the audio input device an audio signal representative of an audio sound input; process the audio signal to detect an indication of a voice activated command included in the audio sound input; and upon detection of the indication of the voice activated command, configure one or more operating parameters of the IVUS imaging system based on a command instruction included in the voice activated command.

With some embodiments of the second embodiment of the at least one machine readable storage device, the second embodiment of an at least one machine readable storage device comprises: wherein the voice activated command comprises a keyword followed by the command instruction.

With some embodiments of the second embodiment of the at least one machine readable storage device, the second embodiment of an at least one machine readable storage device further comprises: instructions that in response to being executed by the processor cause the processor to: process the audio signal using speech recognition software to detect the indication of a voice activated command included in the audio sound input.

With some embodiments of the second embodiment of the at least one machine readable storage device, the second embodiment of an at least one machine readable storage device further comprises: instructions that in response to being executed by the processor cause the processor to: perform an imaging procedure using the IVUS imaging system, the imaging procedure performed based at least in part on the one or more operating parameters configured based on the command instruction included in the voice activated command.

Embodiments of the present disclosure may be implemented as a second method for an intravascular ultrasound (IVUS) imaging system, the second method comprising: receiving, from an audio input device coupled to a computing device, an audio signal representative of an audio sound input; processing the audio signal to detect an indication of a voice activated command included in the audio sound input; and upon detection of the indication of the voice activated command, configuring one or more operating parameters of the IVUS imaging system based on a command instruction included in the voice activated command.

With some embodiments of the second method, the second method comprises: wherein the voice activated command comprises a keyword followed by the command instruction.

With some embodiments of the second method, the second method further comprises: performing an imaging procedure using the IVUS imaging system, the imaging procedure performed based at least in part on the one or more operating parameters configured based on the command instruction included in the voice activated command.

### BRIEF DESCRIPTION OF THE SEVERAL VIEWS OF THE DRAWINGS

To easily identify the discussion of any element or act, the most significant digit or digits in a reference number refer to the figure number in which that element is first introduced.

FIG. 1A illustrates an IVUS imaging system including a control system coupled to a catheter.

FIG. 1B illustrates a cardiac vascular structure and a catheter of an IVUS imaging system positioned within one of the cardiac arteries.

FIG. 1C illustrates a cardiac vascular structure with multiple stents and a catheter of an IVUS imaging system positioned within one of the cardiac arteries.

FIG. 2A illustrates and example series of IVUS image frames.

FIG. 2B illustrates a longitudinal representation of the example series of IVUS image frames of FIG. 2A.

FIG. 3 illustrates the IVUS imaging system according to some embodiments of the present disclosure.

FIG. 4 illustrates a logic flow for selection of an imaging procedure from a listing of pre-programmed procedures using voice activated commands.

FIG. 5 illustrates a logic flow for cancelation of a selection of a selected imaging procedure using voice activated commands.

FIGS. 6A-6B illustrate a logic flow for creating a new imaging procedure using voice activated commands.

FIG. 7 illustrates a logic flow for controlling a visual display being provided on a display device using voice activated commands.

FIG. 8 illustrates a graphical user interface showing a list of pre-programmed imaging procedures that may be accessed by and interacted with using voice activated commands.

FIG. 9 illustrates a graphical user interface showing a selected imaging procedure that may be accessed by and interacted with using voice activated commands.

FIG. 10 illustrates a version of the graphical user interface of FIG. 9 that has been interacted with using voice activated commands.

FIG. 11 illustrates another version of the graphical user interface of FIG. 9 that has been interacted with using voice activated commands.

FIGS. 12A-12B illustrate various stages of a graphical user interface being interacted with using voice activated commands to create a new imaging procedure.

FIG. 13 illustrates an embodiment of a first layout for a graphical display for an IVUS imaging system that may be interacted with using voice activated commands.

FIGS. 14A, 14B, and 14C illustrate versions of the graphical user interfaces using the first layout of FIG. 13 and that can be or have been interacted with using voice activated commands.

FIG. 15A illustrates an embodiment of a second layout for a graphical display for an IVUS imaging system that may be interacted with using voice activated commands.

FIG. 15B illustrates a version of graphical user interfaces using the second layout of FIG. 15A and that can be or has been interacted with using voice activated commands.

FIG. 16 illustrates a graphical user interface showing a navigational bar that can be interacted with using voice activated commands.

FIG. 17 illustrates a computer-readable storage medium.

FIG. 18 illustrates a diagrammatic representation of a machine.

### DETAILED DESCRIPTION

It is to be appreciated by those skilled in the art that the embodiments disclosed may be readily utilized as a basis for modifying or designing other structures for carrying out the same purposes of the present disclosure. The novel features of the disclosure, both as to its organization and operation, together with further objects and advantages, will be better understood from the following description when considered in connection with the accompanying figures. It is to be expressly understood, however, that each of the figures is provided for the purpose of illustration and description and is not intended as a definition of the limits of the present disclosure.

As noted, the present disclosure relates to IVUS imaging systems, and control of the operation of the IVUS imaging systems, using voice activated commands. As an example, voice activated commands may be received as an audio sound input to the system and processed by the IVUS imaging system or a computer system coupled to the IVUS imaging system using speech recognition software and programming, wherein the voice activated commands include one or more command instructions as part of the voice activated command. The command instruction(s) may be used to control one or more operations associated with the IVUS imaging system, including setup procedures for performing imaging procedures and/or for the control of displayed information being provided by the system prior to, during, and/or after performance of procedure(s) utilizing the IVUS imaging system. In determining that an audio sound input received by the IVUS imaging system is a voice activated command, embodiments incorporate speech recognition software or programming configured to detect a keyword, that when included in the audio sound input and provided prior to the command instructions, triggers the speech recognition software or programming to process the subsequent audio inputs as a command instruction. The use of voice activated command capability provides several advantages, including the advantage of hands-free control over the operation of the IVUS imaging system, especially when the IVUS imaging system is being utilized in a sterile field environment.

Suitable IVUS imaging systems include, but are not limited to, one or more transducers disposed on a distal end of a catheter configured and arranged for percutaneous insertion into a patient. Examples of IVUS imaging systems with catheters are found in, for example, U.S. Pat. Nos. 7,246,959; 7,306,561; and 6,945,938; as well as U.S. Patent Application Publication Numbers 2006/0100522; 2006/0106320; 2006/0173350; 2006/0253028; 2007/0016054; and 2007/0038111; all of which are incorporated herein by reference.

Inputting data and/or control inputs into any device in a sterile field environment presents challenges due to the need to avoid direct contact on non-sterile surfaces, which is usually accomplished by using drapes and gloves. Most devices in these environments rely on touch input or external devices such as joysticks, keyboards, and/or a computer mouse for these inputs. However, the presence of drapes and gloves complicates these input interactions. Additionally, external devices, such as pendants and floor devices that are include input devices such as pushbutton and switches, can clutter the workspace.

Further, these manually actuated input devices must be located nearby the user and physically operated to perform their intended functions. Healthcare professionals must input data swiftly and accurately to maintain workflow efficiency and minimize errors when performing various procedures, including procedures being performed using IVUS imaging systems. Therefore, developing intuitive and efficient input methods is crucial to preserving both the sterility and efficiency of medical procedures.

The use of voice activated commands in conjunction with medical devices such as but not limited to IVUS imaging systems, provide a touchless interaction technique configured to limit or eliminate the need for direct touching and/or physical operation of one or more devices, including data input devices such as pushbuttons, foot switches, and even computer keyboards, mouse, and touch screens, thereby reducing the effort required to maintain sterility. Users simply dictate their commands using their voice(s) as an audio sound input to the system, and the system performs the corresponding actions using speech recognition technology. This seamless interaction enhances efficiency and allows healthcare professionals to focus more on patient care rather than navigating through menus on a display screen or device.

In various embodiments, the audio sound inputs to the system are referred to as voice activated commands or as voice commands and consist of a preamble keyword followed by one or more command instructions. The keyword may be pre-programed as a word not normally used in routine verbal communication and therefore is selected and programmed into the system to avoid incorrect detection or accidental triggering of the system when users begin their normal conversations rather than dictations and thus reduce the input error rate. By way of non-limiting example, the preamble keyword “Avvigo” may be used as the trigger word to alert the speech recognition software that a voice activated command is being annunciated and directed to the IVUS imaging system. Once a keyword has been detected in an audio input string received at the IVUS imaging system, the speech recognition software is configured to process the subsequent audio string to detect the presence of one or more command instructions directed to the IVUS imaging system for use in controlling one or more operational aspects of the system. In various embodiments, the speech recognition software is configured to detect command instructions by comparing the word or words included in the audio input string received following the detection of the keyword to a list of pre-programmed command words and command instructions. Once a match has been made between the word or words included in a command instruction and the list of pre-programmed command words or command instructions, the speech recognition software may be configured to output instructions for the control and/or operation of the IVUS imaging system in compliance with the requested control and/or operation(s) associated with the detected command instruction(s).

Throughout the remainder of this disclosure, a voice activated command will be described using the nomenclature “[Keyword], [text of command instruction],” wherein the term “[Keyword]” represents the use of the pre-defined keyword, or any allowed variations thereof, follow by the word(s) or command phrases that identifies the command instruction or command instruction(s). An example of a voice activated command can be represented as follows. A system user can say, “[Keyword], bookmark.” When provided to the speech recognition software of the IVSU imaging system as an audio string, the speech recognition software will detect the presence of the keyword and then process the “bookmark” portion by recognizing “bookmark” as corresponding to or the equivalent of one of a list of pre-program command instruction. As a result, the speech recognition software can be further configurated to output instructions so that the system will place a bookmark at the current cursor position on a display screen.

Embodiments of the IVUS imaging system may include one or more components for effective voice commands, such as:
Headset with Microphone: This is connected to the IVUS imaging system via Bluetooth or USB, allowing for clear voice input.
Voice Training File: A voice training file can be created and utilized by the speech recognition software to ensure that voice commands are clearer and more accurately recognized, especially for users with accents.
Voice Command List: A comprehensive list of available voice commands should be accessible, possibly integrated within the IVUS imaging system software, to guide users on how to effectively utilize the system.

In various embodiments, the set of command words and command instructions that will be recognized by the speech recognition software can be coded or provided in a configuration file. These commands can be crafted and designed to be concise, clearly aligned with their specific functions, and easy to remember. This ensures that users can quickly and efficiently execute tasks without confusion. By way of nonlimiting examples, a voice activated command such as, “[Keyword] Start Recording,” may be used as a control input signal to the IVUS imaging system to start the recording of an imaging procedure, while a voice activated command such as, “[Keyword], Stop Recording,” may be used as a control input signal to the IVUS imaging system to stop the recording of the imaging procedure that is underway at the time.

The use of the voice activated technology in conjunction with an IVUS imaging systems can also accomplish tasks that previously need a sequence of actions now using just one single voice command, thus streamlining the workflow and improving the efficiency. For example, to export a case, users previously had to navigate through multiple sub-menus, select the recorded case, and click the “Export” button. With voice activated commands, users can simply say, “[Keyword], export the current case,” and accomplish exporting the case using just one voice activated command. In another example, to record a 5-minute session, users previously needed to click the “start” button, count down 5 minutes, and then click the “stop” button. With voice activated commands, users can dictate, “[Keyword], record a 5-minute session from now,” and accomplish the timing function using just the single voice activated command. Additional features that may be provided using the voice activated commands as described in this disclosure include:
Voice Accent: If the user has an accent, a training file may be necessary. The training file is created prior to using voice activated commands in the speech recognition software. The training file helps the software interpret accents by pre-training to remove the accent. In one example, one pre-training session takes around 5 minutes, and recognition accuracy improves significantly after the completion of at least two sessions. Users can create their own training profiles and activate them when the user operates the system. When a system/device is shared among multiple people, individual selection of their training profile improves ease of use.
Noise cancellation: A sterile field work environment is often noisy due to constant communication between healthcare professionals and the sounds from medical devices, such as ultrasounds. To address this, embodiments as described in this disclosure can use headsets to cancel out the noise and improve recognition accuracy. The headset may not be part of the sterile field for example when wireless devices are used).
Incorrect Trigger: Incorrect detection or accidental triggering of the system can occur when users start normal conversations instead of dictations. Using a special preamble keyword (such as Avvigo) can help prevent or eliminate these occurrences.
Versatile Matching: Embodiments allow a more intelligent and versatile matching that enables users to use different vocabulary for a single action, improving the user experience to match how people naturally speak. For example, the speech recognition software may recognize that the voice activated commands such as, “[Keyword] start recording” and “[Keyword] begin recording” and “[Keyword] go recording” request the same operation to be performed by the IVUS imaging system.

In the past, the requirements for the sterile field make computer input more difficult. Voice activated commands are one method that can be used to make operation of the IVUS imaging system and the associated clinical software easier to use in a sterile field setting. The use of the voice activated commands as described in this disclosure provide an improvement to the field of operating medical devices, both within and outside of a sterile field environment, including:
Speed and Efficiency: Voice commands can reduce the time and actions needed to perform tasks. Instead of navigating through multiple sub-menus, a simple voice command like “Export the current case” completes the action instantly.
Convenience: Voice commands allow for hands-free operation, which is particularly useful in sterile field and when multitasking or when user hands are occupied.
Accessibility: Voice commands make technology more accessible to users who find it challenging to use traditional input methods (touch screen, keyboard, mouse etc.), especially when they are covered (i.e., under drape).
Reduced Cognitive Load: Users do not need to remember the exact steps or menu paths, which simplifies the process and reduces mental effort.

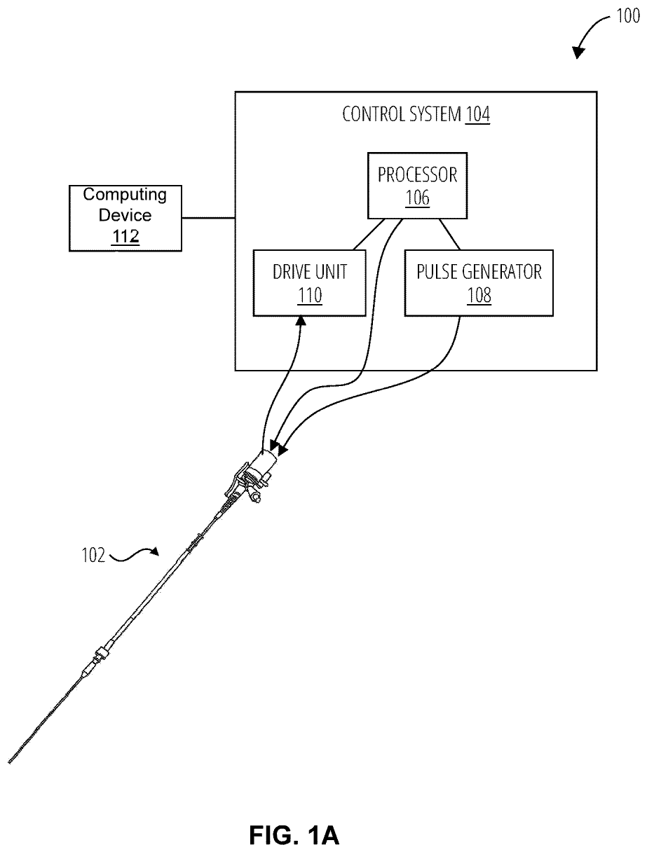

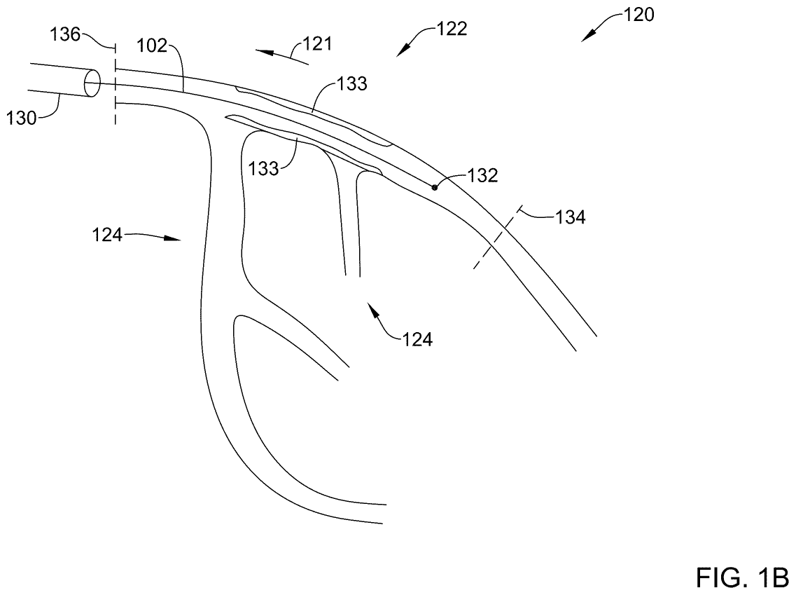

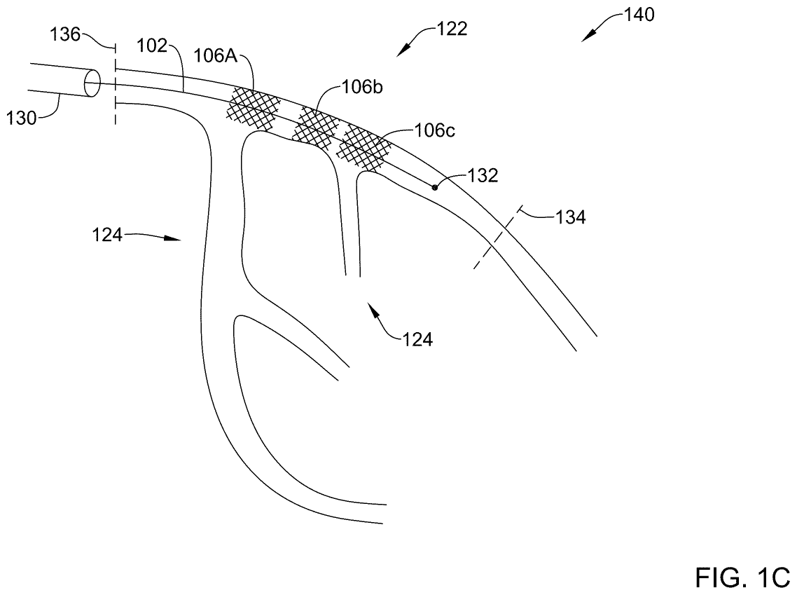

FIG. 1A illustrates an IVUS imaging system 100 including a control system 104 coupled to a catheter 102. The control system 104 may include, for example, a processor 106, a pulse generator 108, and a drive unit 110. The pulse generator 108 forms electric pulses that may be input to one or more transducers (not shown) disposed in the catheter 102. With some embodiments, mechanical energy from the drive unit 110 can be used to drive an imaging core (also not shown) disposed in the catheter 102. In at least some embodiments, electric signals transmitted from the one or more transducers may be input to the processor 106 for processing. In at least some embodiments, the processed electric signals from the one or more transducers can be used to form a series of images, described in more detail below. For example, a scan converter can be used to map scan line samples (e.g., radial scan line samples, or the like) to a two-dimensional Cartesian grid, which can be used as the basis for a series of IVUS images that can be displayed, for example on a tablet and/or a computer monitor or display, and is viewable by a user, including users within the sterile environment where the IVUS imaging system 100 is being utilized.

In at least some embodiments, the processor 106 may also be used to control the functioning of one or more of the other components of the control system 104. For example, the processor 106 may be used to control at least one of the frequency or duration of the electrical pulses transmitted from the pulse generator 108, the rotation rate of the imaging core by the drive unit 110. Additionally, where IVUS imaging system 100 is configured for automatic pullback, the drive unit 110 can control the velocity and/or length of the pullback.

As further described below, control system 104 can be configured to be controlled with respect to one or more operating parameters and/or with respect to various automated procedures, such as automatic pullback procedures, which may be input into the control system 104 using voice activated commands. In some embodiments, control system 104 is configured to receive one or more operating parameters and/or to receive instruction related to operating procedures that have been provided, in full or in part, by use of voice activated commands. In some embodiments, the voice activated commands have been received and processed into instructions by a computing device 112 and provided for example to processor 106 of IVUS imaging system 100 to control operating parameters and/or operating procedures that can be utilized by the IVUS imaging system to perform any of the functions ascribed to the system. In various embodiments, processor 106 can be configured to provide some part of or all the processing required to generate the instructions for operation of the system based on the received voice activated commands.

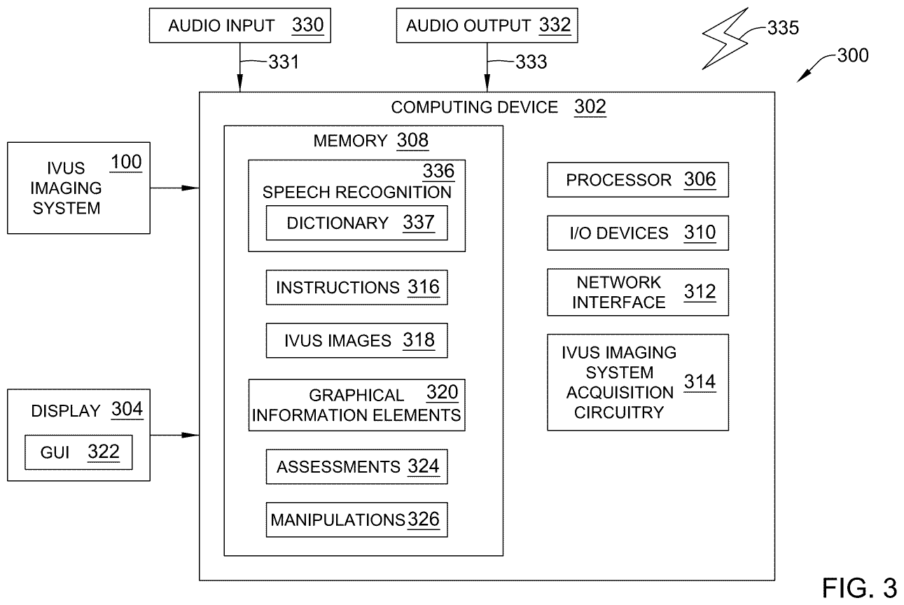

In various embodiments, control system 104 is coupled to or incorporates the computing device 112. Computing device 112 may be an embodiment of computing device 302 as illustrated with respect to FIG. 3 and further described below. Computing device 112 may include any of the devices, software, hardware, and/or firmware and configured to perform any of the function and to provide any of the features ascribed to computing device 302, including imaging system acquisition circuitry, image storage, and speech recognition functions associated with utilization of voice active commands as described herein.

FIG. 1B illustrates a cardiac vascular structure 120 and a catheter 102 of the IVUS imaging system 100 positioned within one of the cardiac arteries. The cardiac vascular structure 120 includes a cardiac artery 122 (e.g., cardiac artery, vessel, or the like) with several side branches 124 (e.g., descending arteries, marginal arteries, vessels, or the like) branching off the cardiac artery 122. IVUS images may be captured at several stages of a percutaneous coronary intervention (PCI), wherein PCI procedures can be guided by intravascular imaging. For example, prior to performing a PCI procedure, during a PCI procedure, and/or after a PCI procedure, an intravascular imaging catheter (e.g., IVUS imaging catheter 102, FIG. 1A) is inserted into the cardiac artery 122 via a guide catheter 130 and images of the cardiac artery 122 are captured. The IVUS imaging catheter 102 includes an ultrasound transducer 132 disposed on a distal end of the IVUS imaging catheter. During the PCI procedure, the IVUS imaging catheter 102 can be pulled back from a distal point 134 in the cardiac artery 122 to a proximal point 136 in the cardiac artery 122, as illustratively represented by arrow 121, and intravascular image frames are captured while the IVUS imaging catheter 102 is pulled back. In some embodiments, the area of cardiac artery 122 included between distal point 134 and proximal point 136 includes something of interest, such as a buildup of plaque 133, wherein the imaging of the artery that is being performed is done to diagnose and/or to determine treatment and/or the efficacy of a treatment performed on the artery.

Control parameters for performing the imaging procedure as depicted in FIG. 1B may be selected and/or entered into the IVUS imaging system by use of voice activated commands. For example, a set of parameters for performing the imaging procedure involving artery structure 120 may have been input into processor 106 using voice activated commands received and processed by computing device 112. In addition, various aspects of visual display(s) being provided before, during, and after the performance of the procedure, for example on a display device such as display 304 (FIG. 3) included as part of computing device 112 may be configured, altered, and manipulated using voice activated commands in order to provide a display of the desired types of information for a user, such as a physician, in a desired format controlled by the use of voice activated commands.

FIG. 1C illustrates a cardiac vascular structure 140 with multiple stents (106a, 106b, and 106c), and a catheter 102 of an IVUS imaging system positioned within one of the cardiac arteries. The cardiac vascular structure 140 includes a cardiac artery 122 (e.g., cardiac artery, vessel, or the like) with several side branches 124 (e.g., descending arteries, marginal arteries, vessels, or the like) branching off the cardiac artery 122. The cardiac vascular structure 140 further depicts stents 106a, 106b, and 106c disposed (or placed) in portions of the cardiac artery 122. It will be appreciated that the stents 106a, 106b, and/or 106c may be placed as part of a PCI procedure (or multiple procedures) to treat a stenosis in the cardiac artery 122. That is, stents 106a, 106b, and 106c can be placed as part of PCI procedure(s) (e.g., stenting balloon dilation, stenting with atherectomy, or the like) intended to dilate or open blocked or partially occluded vessels. In some examples, one of the stents 106a, 106b, and/or 106c can be drug-eluting.

As noted above, PCI procedures can be guided by intravascular imaging. For example, prior to performing a PCI procedure, during a PCI procedure, and/or after a PCI procedure; an intravascular imaging catheter (e.g., IVUS imaging catheter 102) is inserted into the cardiac artery 122 via a guide catheter 130, and images of the cardiac artery 122 are captured. The IVUS imaging catheter 102 includes an ultrasound transducer 132 disposed on a distal end of the IVUS imaging catheter 102. During the post-PCI procedure, the IVUS imaging catheter 102 can be pulled back from a distal point 134 in the cardiac artery 122 to a proximal point 136 in the cardiac artery 122, and intravascular image frames are captured while the IVUS imaging catheter 102 is pulled back.

Control parameters for performing the imaging procedure as depicted in FIG. 1C may be selected and/or entered into the IVUS imaging system by use of voice activated commands. For example, a set of parameters for performing the imaging procedure involving cardiac vascular structure 140 may have been input into processor 106 using voice activated commands received and processed by computing device 112. In addition, various aspects of visual display(s) being provided before, during, and after the performance of the procedure, for example on a display device such as display 304 (FIG. 3) included as part of computing device 112, may be configured, altered, and manipulated using voice activated commands in order to provide a display of the desired types of information for a user, such as a physician, in a desired format controlled by the use of voice activated commands.

For example, the stent segments can be displayed on a GUI, with each stent in each stent segment visually represented, and a user can select a stent segment to interrogate via a stent assessment process. A physician can use this information presented via the GUI to assess the quality and/or effectiveness of the placement of stents 106a, 106b, and/or 106c in the cardiac artery 122. Control over the display format and other aspects of the GUI, such as control over the display of the text data associated with the stent assessment, may be controlled and/or modified by a user using voice activated commands.

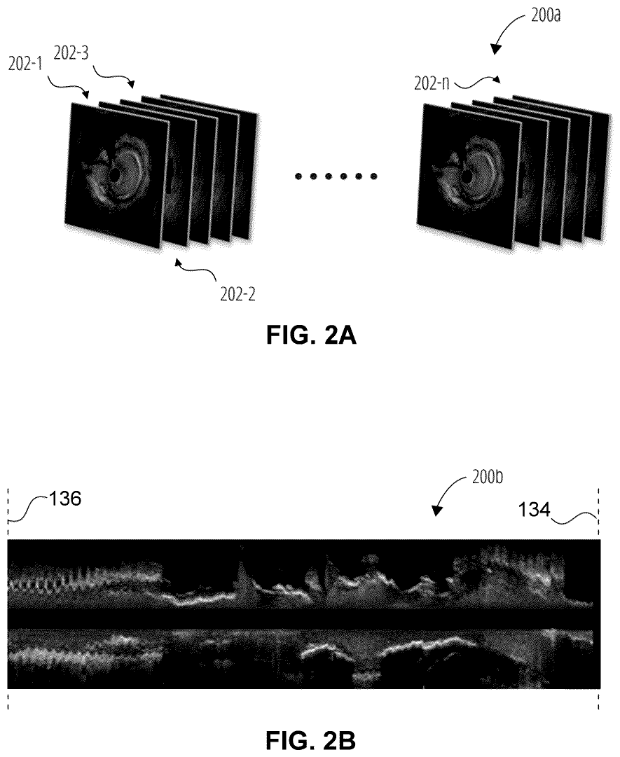

FIG. 2A and FIG. 2B illustrate a series of IVUS image frames 200a and a longitudinal view 200b of the series of IVUS image frames 200a. As described above, several IVUS image frames can be captured while IVUS imaging catheter 102 is moved through cardiac artery 122. For example, FIG. 2A depicts the series of IVUS image frames 200a including IVUS image frames 202-1, 202-2, 202-3 to 202-n, where n is a positive integer, in this case, greater than ten (10). In practice, n can be any positive integer greater than or equal to two (2) but will often be greater than one hundred.

The series of IVUS image frames 200a can be stacked or grouped and depicted longitudinally to represent a longitudinal slice of the cardiac artery 122 from distal point 134 to proximal point 136. For example, FIG. 2B illustrates longitudinal view 200b which shows a longitudinal slice of cardiac artery 122 from distal point 134 to proximal point 136 formed from the series of IVUS image frames 200a. As noted above, a stent or stents (e.g., stents 106a, 106b, and/or 106c) may be represented in the series of IVUS image frames 200a (e.g., where they are captured as part of a post-PCI procedure, or the like).

FIG. 3 illustrates an IVUS imaging system 300, according to some embodiments of the present disclosure. In general, IVUS imaging system 300 is a system for processing, annotating, and presenting IVUS images. IVUS imaging system 300 can be implemented in a commercial IVUS guidance or navigation system, such as, for example, the AVVIGO® Guidance System available from Boston Scientific®. The present disclosure provides advantages over prior or conventional IVUS imaging systems in that the system is configured to control parameter inputs to operational procedures, and to provide control over the operational procedures being performed by the system, using voice activated commands. The use of voice activated commands allows the users of the system to control various operating parameters and procedures of the system without the need to have physical contact with any of the input devices otherwise providing control inputs into the system. In addition, the system is configured to allow control over one or more visual display devices, such as a tablet or a display screen, for example to modify the layout of the graphical indications being displayed and/or to request the display of information in a format that is not currently being provided by the graphical indications displayed, again without the need to have physical contact with any of the input devices associated with the system.

With some embodiments, IVUS imaging system 300 could be implemented as part of control system 104. Alternatively, control system 104 could be implemented as part of computing device 302. As depicted, IVUS imaging system 300 includes a computing device 302 coupled to IVUS imaging system 100. Optionally, IVUS imaging system 300 includes IVUS imaging system 100, computing device 302 and display 304.

Computing device 302 can be any of a variety of computing devices. In some embodiments, computing device 302 can be incorporated into and/or implemented by a console of display 304. With some embodiments, computing device 302 can be a workstation or server communicatively coupled to IVUS imaging system 100 and/or display 304. With still other embodiments, computing device 302 can be provided by a cloud-based computing device, such as, by a computing as a service system accessibly over a network (e.g., the Internet, an intranet, a wide area network, or the like). Computing device 302 can include processor 306, memory 308, input and/or output (I/O) devices 310, network interface 312, and IVUS imaging system acquisition circuitry 314.

The processor 306 may include circuity or processor logic, for example any of a variety of commercial processors. In some examples, processor 306 may include multiple processors, a multi-threaded processor, a multi-core processor (whether the multiple cores coexist on the same or separate dies), and/or a multi-processor architecture of some other variety by which multiple physically separate processors are in some way linked. Additionally, in some examples the processor 306 may include graphics processing portions and may include dedicated memory, multiple-threaded processing and/or some other parallel processing capability. In some examples, the processor 306 may be an application specific integrated circuit (ASIC) or a field programmable integrated circuit (FPGA).

The memory 308 may include logic, a portion of which includes arrays of integrated circuits, forming non-volatile memory to persistently store data or a combination of non-volatile memory and volatile memory. It is to be appreciated, that the memory 308 may be based on any of a variety of technologies. In particular, the arrays of integrated circuits included in memory 308 may be arranged to form one or more types of memory, for example dynamic random-access memory (DRAM), NAND memory, NOR memory, or the like.

I/O devices 310 can be any of a variety of devices to receive input and/or provide output. For example, I/O devices 310 can include, a keyboard, a mouse, a joystick, a foot pedal, a display, a touch enabled display, a haptic feedback device, an LED, or the like. In addition, system 300 can include additional devices, such as audio input devices 330 and audio output devices 332, which enable system 300 to be controlled and operated, at least in part or in full, using voice activated commands. In some embodiments, audio input devices 330 comprise one or more microphones or similar device(s) that are configured to detect audio sounds, such as provided by a human voice, and to convert the audio sounds into a signal, such as an electrical signal, which is communicatively coupled to computing device 302 via communication link 331. In some embodiments, audio input device(s) 330 may be positioned at one or more locations within a room where cardiac procedures utilizing the IVUS imaging system 100 are being performed. In some embodiments, at least one of the audio input device(s) 330 is provided as part of a headset that can be worn by a user, such as a physician, while working in the area where the IVUS imaging system 300 is be utilized.

Communication link 331 may be a wired connection or a wireless connection and may be provided using any suitable protocol and format for transmission of electrical signals, such as Bluetooth. Signals generated by the detection of audio sounds using audio input devices 330 are provided to speech recognition software 336 for processing. Speech recognition software 336 is configured to process the received signals from the audio input devices 330, and to detect the occurrence of a “Keyword” included in the audio sounds, such as “Avvigo”. When a keyword is detected, speech recognition software 336 is further configured to process the audio sounds following the occurrence of the keyword to determine if a proper command string of a word or words followed the keyword. A dictionary 337 may be included in the speech recognition software 336, wherein dictionary 337 includes a list of words and/or phrases that are proper commands that may acted upon by system 300 once a proper command instruction has been received by the speech recognition software 336. In some embodiments, the speech recognition software 336 is configured to compare word(s) and/or phrases that are detected following the detection of a keyword in an audio string provide by the audio input devices(s) 330 and determine if the word(s) and/or phrase(s) included in the audio string correspond to a proper command instruction. If so, speech recognition software 336 may be configured to generate and to output instructions that are provided to one or more other components included in system 300, such as IVUS imaging system 100 and/or display 304, to configure and/or control the operation of these devices.

Speech recognition module may in some embodiments be configured to generate output instructions that include controls for audio output devices 332, to cause audio output devices 332 to provide an audible output, such as words, beeps, or chime sounds, that are configured to provide an output indication to one or more users present in the area where the system 300 is being utilized. For example, speech recognition software 336 may provide output instructions to cause audio output devices 332 to provide an audio word or phrase, such as “confirmed,” in response to receiving an audio input from the audio input devices 330. In various embodiments, audio output device(s) 332 comprise one or more speakers located at one or more positions within the area where system 300 is being utilized. Audio output device 332 may be coupled to computing device 302 using wired connections, wireless connections, or a combination of wired and wireless connections, using any appropriate communication protocol and format, as illustratively represented by communication link 333. Lightning bolt 335 is included in FIG. 3 to represent that either or both of communication links 331 and 333 may be wireless connections.

In various embodiments, speech recognition software 336 is configured to utilize machine learning, such as but not limited to neural networks, to learn variations of the audio inputs, such as user's pronunciation of various versions of the keywords and command words or phrases, based for example on the user's accents and/or volume levels of their annunciated voice outputs. In various embodiments, dictionary 337 included with speech recognition software 366 may include a synonym dictionary that allows synonyms for listed command words that may occur in audio strings received by audio input devices 330 to be processed as proper voice activated commands. For example, dictionary 337 may include a listing of words, such as “show” or “show me” that are interpreted to be a proper voice activated command word equivalent to the word “display.” Using the synonyms list, speech recognition software 336 can provide the required operations, such as procedures and/or display operations, even when a user does not use the exact correct command word(s) when requesting an action to be taken by system 300, thereby easing the burden and protentional frustration on the part of the user in his or her interactions with the system.

Embodiments of speech recognition software 336 may incorporate a purchased speech recognition software application, custom programed software applications, or a combinate of purchased and custom programmed software applications. At a high level, the speech recognition software is configured to operate using an algorithm comprising the steps of: monitoring an input signal representative of audio speech detected at an audio input device, detecting the presence of a keyword in the monitored input signal, processing the subsequent portion of the monitored input signal to determine if the input signal includes representations of recognized command words or instructions, and if proper command words or instruction are detected in the monitored input signal, generating and outputting control commands to one or more other devices included in the imaging system, the control commands are configured to control one or more aspects of the operation of these other device(s). In various embodiments, speech recognition software 336 is configured to provide a time out function that is configured to ignore word, phrases, and/or command instructions that are received in the monitored input signal after the detection of a keyword but also after the expiration of a threshold time limit. For example, speech recognition software 336 may be configured to ignore words, phrases, and/or command instructions that are received in the monitored input signal but that occur after a predetermine time limit, such as 10 seconds, following the time of the detection of the keyword. The process of ignoring any additional words, phrases and/or command instructions may continue until the time that another and subsequent keyword is detected in the monitored input signal, at which time the threshold time limit is reset.

Computing device 302 also includes network interface 312, which can include logic and/or features to support a communication interface. For example, network interface 312 may include one or more interfaces that operate according to various communication protocols or standards to communicate over direct or network communication links. Direct communications may occur via use of communication protocols or standards described in one or more industry standards (including progenies and variants). For example, network interface 312 may facilitate communication over a bus, such as, for example, peripheral component interconnect express (PCIe), non-volatile memory express (NVMe), universal serial bus (USB), system management bus (SMBus), SAS (e.g., serial attached small computer system interface (SCSI)) interfaces, serial AT attachment (SATA) interfaces, or the like. Additionally, network interface 312 can include logic and/or features to enable communication over a variety of wired or wireless network standards (e.g., 802.11 communication standards). For example, network interface 312 may be arranged to support wired communication protocols or standards, such as, Ethernet, or the like. As another example, network interface 312 may be arranged to support wireless communication protocols or standards, such as, for example, Wi-Fi, Bluetooth, ZigBee, LTE, 5G, or the like.

The IVUS imaging system acquisition circuitry 314 may include circuity including custom manufactured or specially programmed circuitry configured to receive or receive and send signals between IVUS imaging system 100 including indications of an IVUS run, a series of IVUS images, or a frame or frames of IVUS images.

Memory 308 can include instructions 316. During operation, processor 306 can execute instructions 316 to cause computing device 302 to receive (e.g., from IVUS imaging system 100, or the like) a recording of an “IVUS run” and store the recording as IVUS images 318 in memory 308. For example, processor 306 can execute instructions 316 to receive information elements from IVUS imaging system 100 comprising indications of IVUS images captured by catheter 102 while being pulled back through a vessel, which images comprising indications of the anatomy and/or structure of vessel, including vessel walls and plaque. It is to be appreciated that any captured IVUS images can be stored in a variety of image formats or even non-image formats or data structures that comprise indications of imaged vessel. Further, IVUS images 318 includes several “frames” or individual images that, when represented co-linearly can be used to form an image of the vessel, such as, for example, as represented by IVUS images 200a and/or 200b.

Graphical information elements 320 from IVUS images 318 are used to generate a GUI 322 to be displayed on display 304 based on the graphical information elements 230. Processor 306 can further be configured to execute instructions 316 to generate assessments 324 based on IVUS images 318. In general, the assessments can include vessel boundary detection, lumen boundary detection, plaque burden determination, key frame identification, distances between key frames, among other assessments. As such, in some embodiments, graphical information elements 320 can be generated based on IVUS images 318 and assessments 324.

Additionally, processor 306 can be configured to execute instructions 316 to receive manipulations 326 including modifications to assessments 324. For example, processor 306 can execute instructions 316 to receive modifications to vessel and/or lumen boundaries, key frame locations, or the like and store such modifications as manipulations 326. Responsive to receiving manipulations 326, processor 306 can execute instructions 316 to regenerate graphical information elements 320.

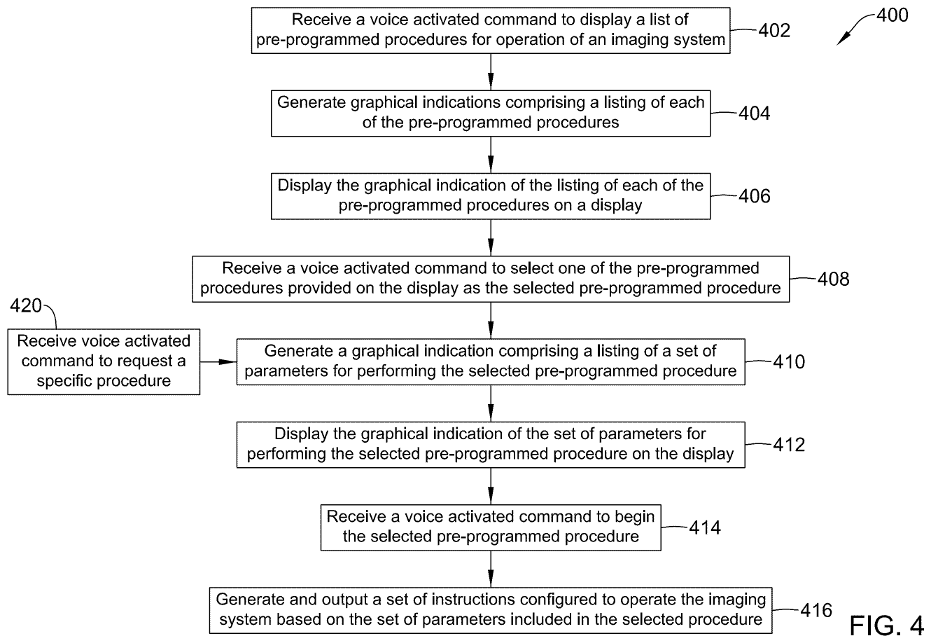

FIG. 4 illustrates a logic flow 400 for selection of an imaging procedure from a listing of pre-programmed procedures using voice activated commands. The logic flow 400 can be implemented by IVUS imaging system 300 and will be described with reference to IVUS imaging system 300 (hereinafter “system 300”) for clarity of presentation. However, it is noted that logic flow 400 could also be implemented by an IVUS guidance or imaging system that is different than system 300.

Logic flow 400 can begin at block 402. At block 402, “receive a voice command to display a list of pre-programmed procedures for operating an imaging system,” causes computing device 302 to retrieve stored data related to a listing of one or more pre-programmed procedures that system 300 is configured to perform. In some embodiments, each of the pre-programmed procedures may be associated with a corresponding procedure identifier, for example a procedure number and a set of one or more operating parameters, each of which may be associated with a value or a range of values for that particular operating parameter. In various embodiments, the set of one or more operating parameters includes operating parameters such as drag speed, drag length, imaging resolution, rotational speed, and associated values or ranges of values for each of the operating parameters that may be required to perform an imaging procedure using system 300.

Logic flow 400 continues at block 404, “generate graphical indications comprising a listing of each of the pre-programmed procedures,” causes computing device 302 to retrieve the listing from stored data related to the listing of the pre-programmed procedures at block 402. In various embodiments, processor 306 may generate the graphical indication as a GUI component that includes a listing of the one or more pre-programmed procedures that system 300 is configured to perform.

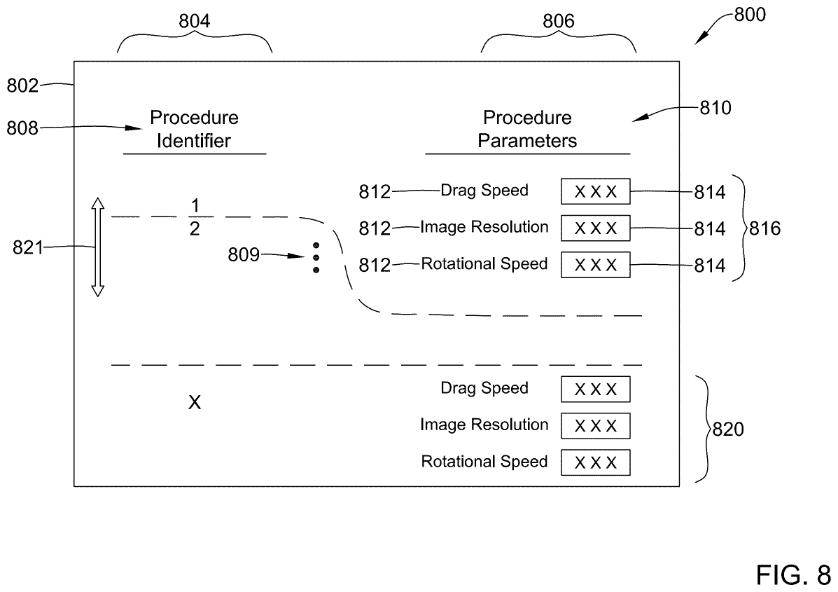

Continuing to block 406, logic flow 400 includes, “display the graphical indication of the listing of each of the pre-programmed procedures on a display,” wherein the GUI component comprising the listing of the one or more pre-programmed procedures is rendered on a visual display 304. For example, as illustrated in FIG. 8, a listing of pre-programmed procedures “1” through “X” may be rendered and displayed on some portion of the visual display 304. The listing associated with each of the one or more pre-programmed procedures may include additional data, such as but not limited to drag speed, drag length, imaging resolution, rotational tool speed, and/or a value or a range of values associated with each one, respectively, of pre-programmed procedures included in the listing.

Having displayed on display 304 the graphical indicators of the pre-programmed list of procedures, logic flow 400 may proceed to block 408, including, “receive a voice activated command to select one of the pre-programmed procedures provided on the display as the selected procedure.”

In response to receiving the voice activated command at block 408, logic flow 400 continues at block 410, “generate a graphical indication comprising a listing of the selected procedure.” In various embodiments, processor 306 may generate the graphical indications as a GUI component that includes a listing of the selected procedure. The graphical indications included in the listing of the selected procedure may include a graphical indication of a procedure identification, such as a number, that is associated with and uniquely identifies the select procedure, along with a one or more values associated with the parameters required for performing the selected procedure.

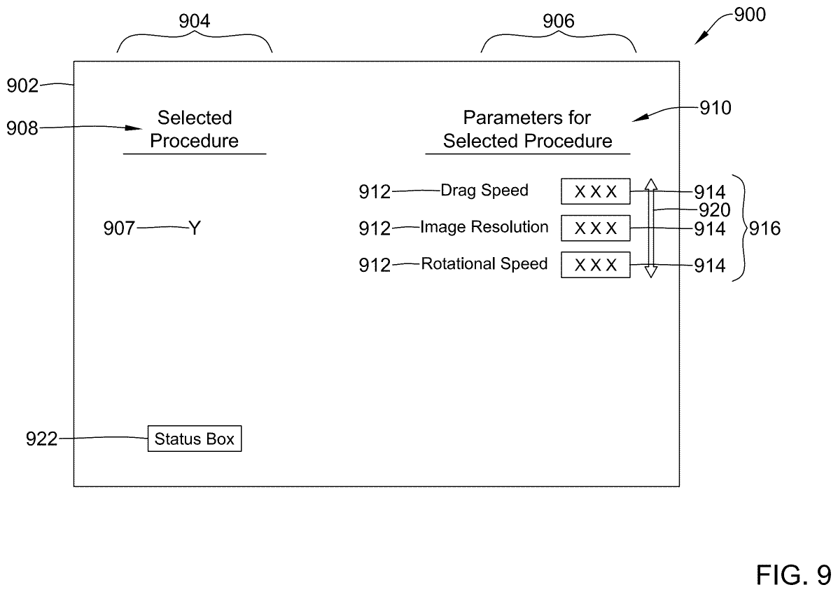

Continuing to block 412, logic flow 400 includes, “display the graphical indication of the set of parameters for performing the selected procedure on the display,” wherein the GUI component comprising the listing of the selected procedure is rendered on visual display 304. For example, as illustrated in FIG. 9, a listing of the procedure identifier and the associated parameters and parameter values are displayed on visual display 304. The listing associated with the selected procedure may include parameters and parameter values, such as but not limited to drag speed, drag length, imaging resolution, rotational tool speed, and/or a value or a range of values associated with each one, respectively, of the selected procedure. Display of the graphical indications at block 412 may also include a graphical field asking for a response confirming that the selected procedure should be started.

As an alternative embodiment, a user may not want to go through each of the steps of logic flow 400 depicted at blocks 402-408 to arrive at a display of parameters of a specific procedure. For example, a user may already know the procedure that they want to utilize, and may request that specific procedure be displayed, for example using a voice activated command, such as “[Keyword], show me procedure Y,” or “[Keyword,] show me the procedure for patient X,” or similar. In response to receiving a voice activated command to select a specific procedure (block 420) directly, embodiments of logic flow 400 can be configured to proceed from block 420 to block 410, wherein at block 410 the parameters for the particular procedure called out by the user are displayed as the “selected” procedure.

Having displayed on display 304 the graphical indicators for the selected or called out particular procedure, logic flow 400 may proceed to block 414, “receive a voice activated command to begin the selected procedure.”

In response to receiving the voice activated command at block 414, logic flow 400 continues at block 416, “generate and output a set of instructions configured to operate the system based on the set of parameters included in the selected procedure.” In various embodiments, the generated set of instruction can be provided to an imaging control system, such as control system 104 (FIG. 1), which in turn causes the control system 104 to proceed with an imaging operation, such as but not limited to an imaging operation illustrated and described with respect to FIG. 1B or with respect to FIG. 1C. In addition, the generated set of instruction can also provide instructions for control of display 304 so that the visual display being provided at display 304 is coordinated with the desired types and format of imagery associated with the operation of system 300 while performing the selected imaging procedure, which in various embodiments includes a visual indication provided on the display 304 showing that an imaging procedure based on the selected procedure is underway.

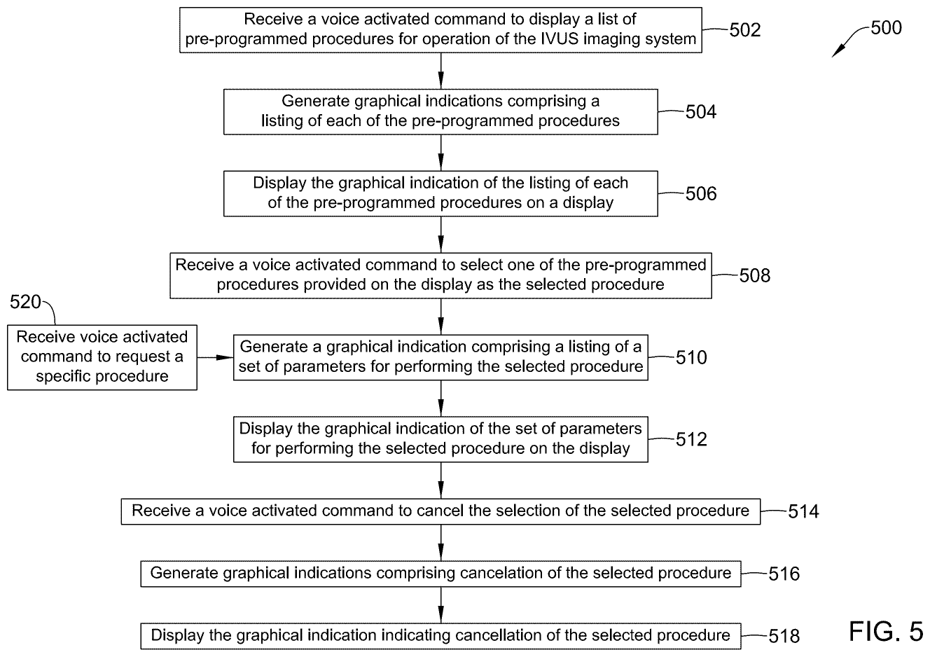

FIG. 5 illustrates a logic flow 500 for cancelation of a selection of a selected imaging procedure using voice activated commands. Logic flow 500 includes a method for using voice activated commands to cancel out of a selected procedure that is being displayed on display 304. Logic flow 500 includes embodiments of a method for controlling the display of information related to the selection and then cancelation of the selection of a set of parameters for performing an imaging procedure using the IVUS imaging system using voice activated commands. Non-limiting examples of the formats for and the type of information that may be displayed and controlled using voice activated commands as part of logic flow 500 are illustrated and described below with respect to FIGS. 8-9 and 11.

Referring to FIG. 5, logic flow 500 includes several initial steps that correspond to the steps described above with respect to FIG. 4 and logic flow 400, including: “receiving a voice activated command to display a list of pre-programmed procedures for operation of the IVUS imaging system” (block 502, corresponding to block 402 of FIG. 4); “generate graphical indications comprising the listing of each of the pre-programmed procedures” (block 504, corresponding to block 405 of FIG. 4); “display the graphical indications of the listing of each of the pre-programmed procedures on a display” (block 506, corresponding to block 406 of FIG. 4); “receive a voice activated command to select one of the pre-programmed procedures provided on the display as the selected procedure” (block 508, corresponding to block 408 of FIG. 4); “generate a graphical indication comprising a listing of a set of parameters for performing the selected procedure” (block 510, corresponding to block 410 of FIG. 4); and “display the graphical indications of the set of parameters for performing the selected procedure on the display” (block 512, corresponding to block 412 of FIG. 4). In various embodiments, one or more of the functions performed by blocks 502-512 of logic flow 500 may include any of the features and functions described above with respect to blocks 402-412 and FIG. 4.

As an alternative embodiment, a user may not want to go through each of the steps of logic flow 500 depicted at blocks 502-508 to arrive at a display of parameters of a specific procedure. For example, a user may already know the procedure that they want to utilize, and may request that specific procedure be displayed, for example using a voice activated command, such as “[Keyword], show me procedure Y,” or “[Keyword,] show me the procedure for patient X,” or similar. In response to receiving a voice activated command to select a specific procedure (block 520) directly, embodiments of logic flow 500 can be configured to proceed from block 520 to block 510, wherein at block 510 the parameters for the particular procedure called out by the user are displayed as the “selected” procedure.

Embodiments of logic flow 500 may begin at block 512 or may arrive at block 512 using one or more of the steps described above with respect to blocks 502-510. From block 512, wherein a visual display is provided including a graphical indication of the set of paraments for performance of the selected procedure, logic flow 500 can proceed to block 514, including, “receive a voice activated command to cancel the selection of the selected procedure.” In response to receiving the voice activated command including a request to cancel the selection of the selected procedure, logic flow 500 may proceed to block 516, which includes, “generate graphical induction comprising a notification of the cancellation of the selected procedure.” Logic flow 500 may then proceed to block 518 including, “display of the graphical indication including the notification of the cancelation of the selected procedure.” Embodiments of logic flow 500 may incorporate an additional feature at blocks 516 and 518 that include generation of an indication that a confirmation of the cancelation is required, wherein the confirmation of the cancellation may also be made using a voice activated command.

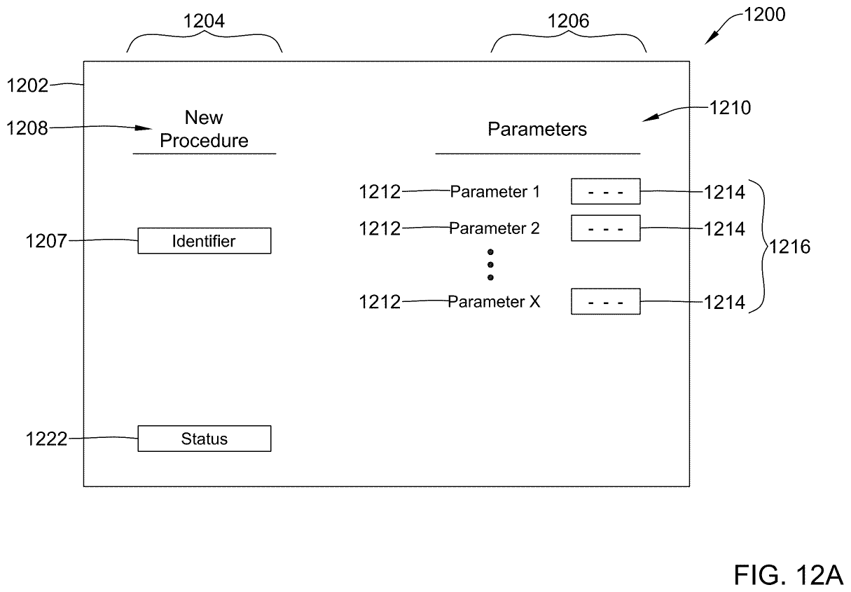

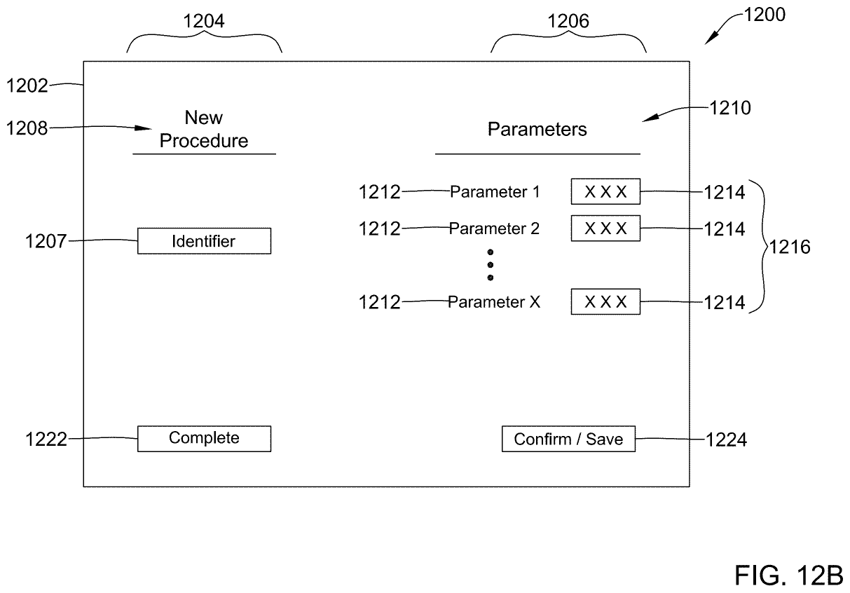

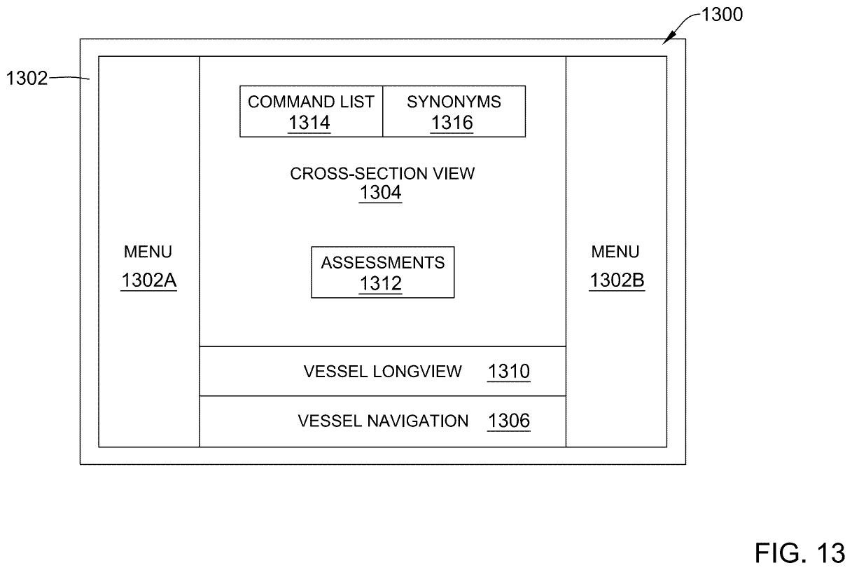

In some embodiments of logic flow 500, cancellation of the selected procedure may include logic flow 500 proceeding, either automatically or based on receipt of another command instruction included in a voice active command, to a different display screen being provided as the visual display. For example, logic flow 500 at block 512 may include proceeding to a display of a listing of the available procedures and associated parameters, for example as shown for visual display 802 in FIG. 8. For another example, logic flow 500 at block 512 may include proceeding to generate and display of a visual display configured to allow creation of a new procedures and entry of data for a set of parameters and parameter values associated with the new procedure, for example as shown for visual display 1202 in FIGS. 12A-12B.

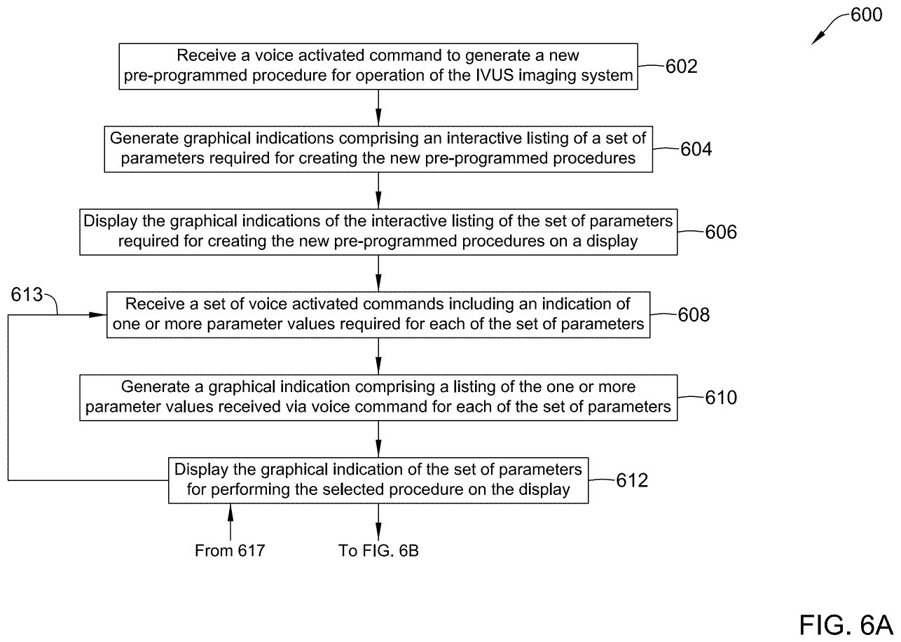

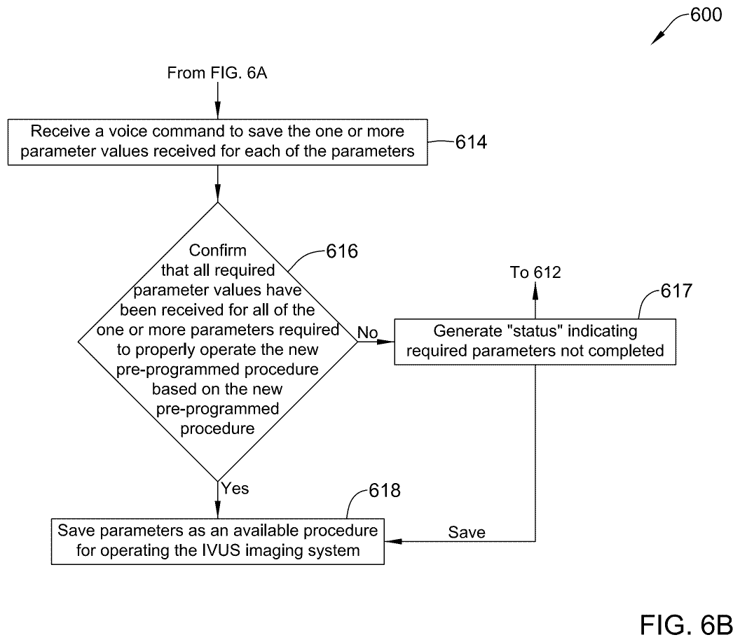

FIGS. 6A-6B illustrate a logic flow 600 for creating a new imaging procedure using voice activated commands. Logic flow 600 includes embodiments of a method for controlling a process for creating a new procedure that may be performed by the IVUS imaging system, and for entering data for a set of parameters associated and/or required for the performance of the newly created procedure, using voice activated commands.

Embodiments of logic flow 600 begin at block 602, including, “receive a voice activated command to generate a new pre-programmed procedure for the operation of the IVUS imaging system.” In one alternative, logic flow 600 may be initiated at block 602 by virtue of the cancelation of a previously selected procedure, such as described above with respect to logic flow 500 and FIG. 5. In another alternative, logic flow 600 may be initiated at block 602 by virtue of a voice activated command received at the IVUS imaging system requesting the modification of one or more parameters associated with a pre-programmed procedure.

Once at block 602, logic flow 600 can proceed to block 604, including, “generate graphical indications comparing an interactive listing of a set of parameters required for creating the new pre-programmed procedure.” Logic flow 600 can then proceed to block 606, including, “display the graphical indications of the interactive listing of the set of parameters required for creating the new pre-programmed procedure on a display.”

Once the graphical indications of the listing of the set of parameters required for the newly created procedure are displayed as described for block 606, logic flow 600 can proceed to block 608, including, “receive a set of voice activated commands including an indication of one or more parameter values required for each of the set of parameters,” for the newly created procedure.

The received voice active commands may include a value or a range of values for one of the set of operating parameters, or a plurality of values and/or ranges of values for more than one of the sets of operating parameters, including all the values for the set of operating parameters for the newly created operating procedure.

In response to receiving the voice activation commands at block 608, logic flow 600 proceeds to block 610, “generate a graphical indication comprising a listing of one or more parameter values received via voice activated commands for each of the set of parameters,” followed by execution of block 612, “display the graphical indication of the set of set of parameters for performing the new pre-programmed procedure on the display,” which includes a updated display of any received parameter values for the set of parameters. In various instances, the generation and display of the graphical indications representing the set of parameters and any parameter values already received, (blocks 610 and 612), may be perform prior to having received parameter value for all the parameters included in the set of parameters listed and/or required to operate the new pre-programmed procedure. In other instances, the generation and display of the graphical indications representing the set of parameters and any parameter values already received, (blocks 610 and 612), may be perform after having received all the parameter values for each of the parameters included in the set of parameters listed and/or required to operate the new pre-programmed procedure.

Regardless of whether all the parameter values for each of the parameters included in the set of parameters have been received or not, logic flow 600 can proceed to block 614, including, “receive a voice activated command to save the one or more parameter values received for each of the parameters,” which are associated with the new pre-programmed procedure.

Upon receiving the voice activated command to save the parameters values at block 614, logic flow 600 can proceed to decision block 616, wherein the IVUS imaging system is configured to review the newly created operating procedure, and to confirm that parameter values for each of the required operating parameters of the newly created procedure have been provided. If the system determines that not all the parameter values have been provided, (the “NO” branch exiting from decision block 616), the logic flow 600 may generate and display a message indicating that not all the required parameter values have been received (block 617), which may include a visual indication showing which parameter(s) have not been set up with acceptable parameter valuers(s). Embodiments of logic flow 600 may then return to block 612. After returning to block 612, additional voice activated commands may be received that include additional parameter value(s) for one or more of the parameters, and the process of generating graphical indications (block, 610) and displaying the graphical indications with the updated parameter value(s) (block 612) may be repeated any number of times, as illustratively indicated by arrow 613 extending from block 612 to block 608.

In response to the status indication at block 617 indicating that not all the parameter values for each of the parameters in the set of parameters has been received, a repeat of the voice command at 614 requesting that the parameter values be saved, in effect “as is,” may be received, in which case logic flow 600 proceeds to block 618.

In the alternative, at decision block 616 if the system determines that all the values for the required parameters have been received, (the “YES” branch extending from decision block 616), then logic flow 600 proceeds to block 618, including, “save parameters as an available procedure for operating the IVUS imaging system.” Embodiments of logic flow 600 at block 618 may include outputting as a visual display an indication that the new pre-programmed procedure has been saved.

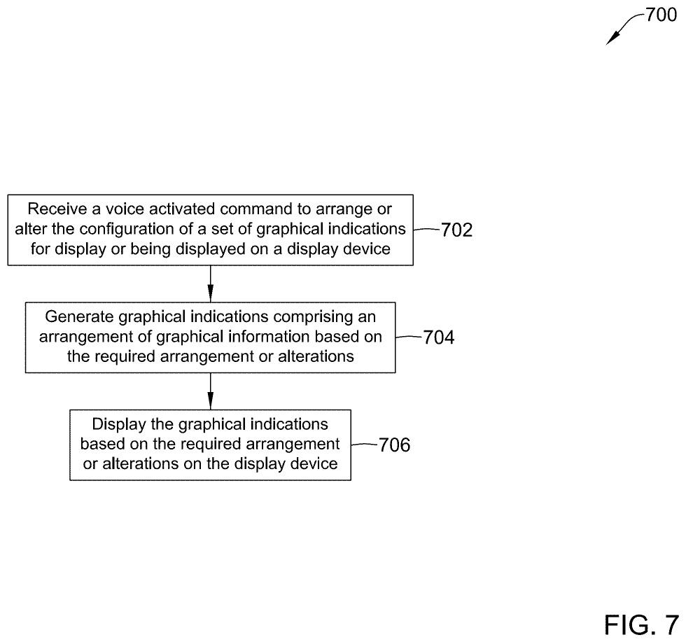

FIG. 7 illustrates a logic flow 700 for controlling a visual display being provided on a display device using voice activated commands. Logic flow 700 includes a method for controlling the display of information, such as information displayed on display 304, including but not limited to manipulation of the layout of the displayed information and/or control over the types and format of the information being displayed. Non-limiting examples of the formats for and the types of information that may be displayed and controlled using voice activated commands is illustrated and described below with respect to FIGS. 8-11, 12A-12B, 13, 14A-14C, 15A-15B, and 16.

Referring again to FIG. 7, logic flow 700 begins at block 702, including, “receive a voice activated command to arrange or alter the configuration of a set of graphical indications for display or being displayed on a display device.” The voice activated command can include the use of a keyword as described above, followed by any of several command instructions configured to control the information being displayed on a display device, such has display 304. Embodiment of these command instructions may include a generic type of command, such as, “[Keyword], display previous screen,” or something as simple such as, “[Keyword] back,” or similar. These voice activated commands may be used to cause the display to go back to and display a screen that was previously displayed. Corresponding voice activated commands may be used in a similar way to cause the display to proceed forward to a display screen previously displayed but was backed away from by a previously received voice activated command.

In some embodiments, the voice activated command may be arranged to cause the display device to display specific information. For example, a voice activated command such as, “[Keyword], display a listing of procedures,” or similar, can cause the processor 306 to generate a listing of available pre-programmed procedures that may be performed by system 300, and which are then displayed on the display device 304. In another example, a voice activated command such as, “[Keyword], display a listing of commands,” or similar, can cause the processor 306 to generate a listing of available commands that would be recognized by system 300, for example using speech recognition software 336, and that may then be acted upon in the appropriate manner by system 300.

In some embodiments, the voice activated command may be arranged to change the layout and/or the format of the graphical information being displayed or to be displayed on the display 304. For example, a voice activated command may include a keyword followed by a command instruction to show or to hide various information, such as menus, image views, assessment information, and/or listings that is/are currently being displayed on display 304, or that a user wants to have displayed on the display. In some embodiments, voice activated commands may include a keyword followed by a command instruction requesting that a certain portion or a particular feature image being displayed on display 304 be enlarged or reduced in size. In some embodiments, a voice activated command may include instructions to enlarge or reduce the size of text being displayed by display 304, resulting in a change in the text size in the that is provided on the display.

The examples of the types of voice active commands that may be received and processed to control the graphical information being displayed by one or more display devices, such as display 304, are not provided in a limiting manner. The types of commands that may be received and processed by system 300 may include any commands and variations thereof that system 300 is programmed to receive, process, and act upon.

In response to receiving a voice activated command at block 702, embodiments of logic flow 700 proceed to block 704, “generate graphical indications comprising an arrangement of graphical information based on the required arrangement or alteration,” as provided in the command instruction(s) included in the voice activated command. Logic flow 700 may then proceed to block 706, including, “display the graphical indications based on the required arrangement or alterations on the display device,” such as display device 304 (FIG. 3).

FIG. 8 illustrates a graphical user interface 800 showing a list of pre-programmed imaging procedures that may be accessed by and interacted with using voice activated commands. The graphical user interface 800 shows at least a portion of a visual display 802 that may be provided in response to a voice activated command received by an IVUS imaging system. For example, the visual display 802 can be generated by IVUS imaging system 300 as GUI 322 and displayed on display device 304. In some embodiments, visual display 802 may be provided by a tablet or other display device. The visual display 802 as shown in FIG. 8 may be provided on a display device in response to a voice activated command, such as, “[Keyword], display a listing of available imaging procedures,” or a similar command, received at the IVUS imaging system. As shown in FIG. 8, visual display 802 includes a first column 804 of information including a first header 808 reciting “Procedure Identifier,” and a listing of procedure numbers “1” through “X” arranged vertically below the first header 808. The number of procedure identifiers that may be included in the listing below the first header 808 is not limited to any number and may include any positive integer number of procedure identifiers “1” through “X”, as illustratively represented by the vertical row of dots 809.

Visual display 802 further includes a second column 806 of information including a second header 810 reciting “Procedure Parameters,” and including a listing of parameter titles 812 and associated parameter value boxes 814 arranged vertically below the second header 810. For each of the procedure identifiers listed in the first column 804, the second column 806 includes a corresponding listing of one or more parameters associated with the procedure. For example, a listing of parameters under second header 810, as generally indicated by bracket 816, is associated with procedure “1” from the first column 804. A listing of parameters under second header 810, as generally indicated by bracket 820, is associated with procedure “X.” It would be understood that for any addition procedures that are listed under first header 808, a corresponding set of parameters can be listed in the second column 806 under the second header 810 and adjacent horizontally to the corresponding procedure number listing.

For each of the listed procedure identifiers, the number and type of parameters listed under column second column 806 is not limited to a particular type and/or to a particular number of parameters. The number and type of parameters listed for each of the procedures may be pre-determined to allow a user viewing the visual display 802 to choose the best or a desired procedure from the listing of procedure identifiers. By way of non-limiting example for procedure 1, a listing of the associated parameters can include parameter titles 812 indicated as “drag speed,” “imaging resolution,” and “rotational speed,” respectively. Each of these parameter titles 812 is positioned horizontally adjacent to a respective set of parameter value boxes 814. For example, each of the parameter value boxes 814 can display a value, for example a numerical value, that is associated with the parameter title 812 that is horizontally adjacent to that box. For example, the parameter value box 814 adjacent to the “drag speed” title may display a numerical value for the drag speed to be used when performing a procedure indicated as procedure identifier “1” in the listing, the parameter box adjacent to the “rotational speed” title may display a value for the rotational speed of the imaging transducer that is used to provide the imaging performed as part of procedure identifier 1 in the listing, and the parameter box adjacent to the “rotational speed” title may display a value for the rotational speed of the imaging transducer that is used to provide the imaging as part of procedure identifier 1 in the listing.

Once visual display 802 has been generated and is being displayed on a display device, additional voice activated commands, for example audio commands spoken by a user such as a physician, may be received at IVUS imaging system, which allows the system to take various actions, including the generation and display of different graphical information based on the received voice activated command(s). For example, a voice activated command such as, “[Keyword], select procedure X,” or similar, may be received at system 300. In response, system 300 may proceed to generate and display graphical information specifically related to the selected procedure, for example as illustrated and described below with respect to FIG. 9.

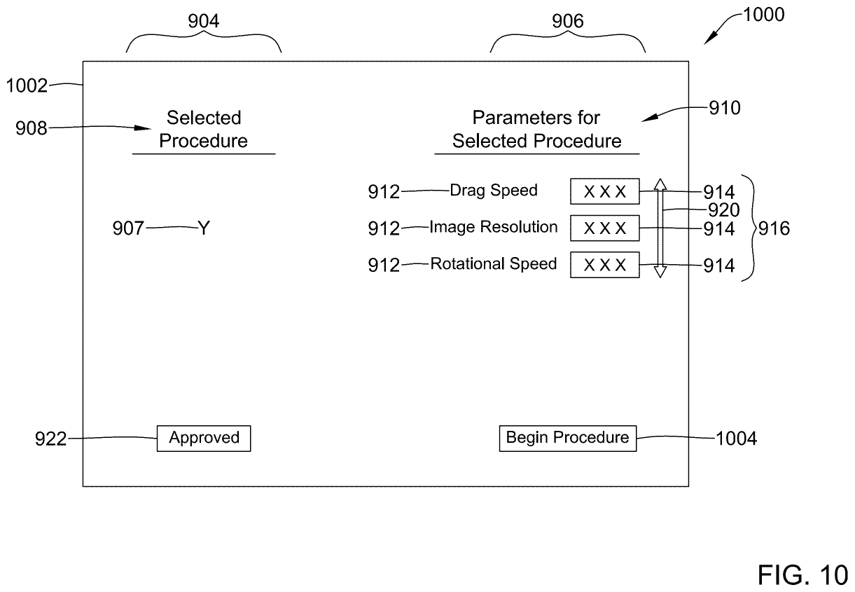

Additional examples can include receiving a voice activated command at system 300, such as, “[Keyword], configure system to perform procedure X,” or similar, which both selects a procedure from the listing of procedure identifiers and requests that the imaging system be configured to perform a procedure or procedures based on the parameters including in the selected procedure, all by using just a single voice activated command. Such a voice activated command can allow system 300 to proceed to generate and display graphical information related to various parameters that will be used by the imaging system as part of proceeding with operations based on the selected procedure, for example as illustrated and described below with respect to FIG. 10.

Additional voice activated commands may include commands configured to sort and/or display procedures that comply with particular criteria. For example, a voice activated command such as, “[Keyword], display only procedures having a [parameter] [modifier] [value],” or similar, wherein “parameter” represents one of the displayed procedure parameters, “modifier” can be a quantifier such as “less than,” “greater than,” or “within a range from,” or similar, and “value” can be a numerical value or value range for the parameter. A non-limiting example can be, “[Keyword], display only procedures having a drag speed in a range from X to Y [measurement units],” or similar. In response to these types of voice activated commands, the listing of procedure identifiers and procedure parameters displayed as part of visual display 802 may be regenerated and displayed including only procedures that conform to the requirements dictated by the received voice activated command. A voice activated command may then be received at system 300 to indicate a selection of the one of the now displayed procedures being provided by visual display 802.

Additional voice activated commands may include voice activated commands configured to modify one or more parameters of an indicated one of the procedures being displayed on visual display 802. For example, a voice activated command such as, “[Keyword], modify the [parameter] for procedure X,” or similar, may be received at system 300, wherein “parameter” represents one of the displayed procedure parameters associated with the indicated procedure identifier associated with “X.” In response to these types of voice activated commands, the visual display 802 may be regenerated and displayed to include graphical information as illustrated and described below with respect to FIGS. 12A-12B, which allow for use of voice activated commands to modify and/or to create a new sets of parameters for a selected procedure.

In embodiments wherein the listing of procedure identifiers and associated procedure identifiers does not fit entirely on the graphical area allocated for visual display 802, a scroll bar 821 may be provided as part of the visual display. When scroll bar 821 is provided, voice activated commands, such as, “[Keyword], scroll up,” and “[Keyword], scroll down,” may be provided to system 300 in order to scroll the listing up or down in order to display additional procedure identifiers and associated parameters as part of visual display 802.

FIG. 9 illustrates a graphical user interface 900 showing a selected imaging procedure that may be accessed by and interacted with using voice activated commands. The graphical user interface 900 shows at least a portion of a visual display 902 that may be provided in response to a voice activated command received by an IVUS imaging system. For example, visual display 902 can be generated by IVUS imaging system 300 as GUI 322 and displayed on display device 304. In some embodiments, visual display 902 may be provided by a tablet or other display device. The graphical display as shown in FIG. 9 may be provided on a display device in response to voice activated command, such as, “[Keyword], select procedure “X”,” or similar, received at the IVUS imaging system, in response to the display of a listing of available procedures, for example as illustrated in FIG. 8, wherein “X” represents a procedure indicator, such as a procedure number, for the particular procedure that was selected using the voice activated command.

As shown in FIG. 9, visual display 902 includes a first column 904 of information including a first header 908 labeled “Selected Procedure,” and a selected procedure indicator 907 “Procedure Y” arranged vertically below the first header 908. Visual display 902 further includes a second column 906 of information including a second header 910 reciting “Parameters for Selected Procedure,” and a listing of parameter titles 912 and associated parameter values boxes 914 arranged vertically below the second header 910. For the procedure identifier listed in the first column 904, the second column 906 includes a corresponding listing of one or more parameters associated with the procedure. For example, a listing of parameters under second header 910, as generally indicated by bracket 916, is associated with procedure Y from the first column.

In instances where the listing of the parameters associated with the selected procedure do not all fit within the area of the display allocated for visual display 902, a scroll bar 920 may be provided as part of the visual display, In some embodiments, voice activated commands, such as, “[Keyword], scroll up,” and “[Keyword], scroll down,” may be provided by a user, and when received and recognized by the system 300, may allow the display to scroll up and scroll down, respectively, to display addition parameters within the display as provided by visual display 902.

In some embodiments, the visual display 902 includes status box 922. Status box 922 can be configured to include a message representative of the status of the selected procedure displayed in the first column 904. For example, the message displayed in the status box 922 may recite, “confirm selection of procedure,” when a confirmation has not yet been received regarding choice of the selected procedure being represented in visual display 902. In some embodiments, a voice activated command may be used, such as, “[Keyword], confirm the choice of selected procedure,” or similar, may be provided to change a status of the system from waiting for confirmation of the selected procedure to for example a ready state for running a procedure using the IVUS imaging system and using the selected and now confirmed procedure, as displayed for example as illustrated and described below with respect to FIG. 10.

In some instances, a user such as a physician may want to proceed with the selected procedure as illustrated by visual display 902, but for example with one or more changes or added parameters for running the procedure. In some embodiments, a voice activated command such as, “[Keyword], change value for parameter X,” or for example, “[Keyword] add additional parameter Z to parameter list for procedure,” may be received at the system. In response to such a command, an interactive version of the visual display, such as illustrated and described with respect to FIG. 12A-12B, may be activated to allow the user to modify one or more of the parameters and/or the associated parameter values, and to save the modifications in some embodiments as a new procedure, as further described below.

FIG. 10 illustrates a version of the graphical user interface of FIG. 9 that has been interacted with using voice activated commands. FIG. 10 is an illustration of a graphical user interface 1000 of at least a portion of a visual display 1002 that may be provided in response to a voice activated command received by an IVUS imaging system. Embodiments of visual display 1002 may be provided on display device 304 in response to a voice activated command received at system 300 indicating approval of the selected procedure previously being displayed on display device 304, such as the visual display 902 as illustrated and described above with respect to FIG. 9.

Referring to FIG. 10, visual display 1002 includes many of the same elements illustrated and described above with respect to FIG. 9, including headings 908 and 910, a procedure indicator 907 indicating the selected procedure, and a display of a set of parameters and parameter values, as generally indicated by bracket 916. FIG. 10 and visual display 1002 differ from FIG. 9 and visual display 902 in that in FIG. 10, the status box 922 now includes an indication, such as the word “APPROVED,” or similar, along with the addition of activation selection box 1004. Activation selection box 1004 may include text, such as, “BEGIN PROCEDURE,” or similar. When displayed, system 300 may be configured to receive a voice activated command, such as, “[Keyword], begin procedure,” or similar, that can be used to start the imaging procedure using the IVUS imaging system and controlled based on the parameter set displayed for the selected procedure in visual display 1002.

In embodiments where the selected procedure includes manual operation, other parameters, such as imaging resolution and/or rotational speed of the imaging, may be controlled based on the set of parameters associated with the selected procedure as the selected procedure is being performed. In embodiments where the selected procedure includes automatic operation, such as automatic pullback, in addition to one or more parameters associated with the procedure related to imaging, one or more parameters related to the movement of the image transducer, such as the drag speed and/or the drag length, may be controlled based on the set of parameters associated with the selected procedure as the selected procedure is being performed.

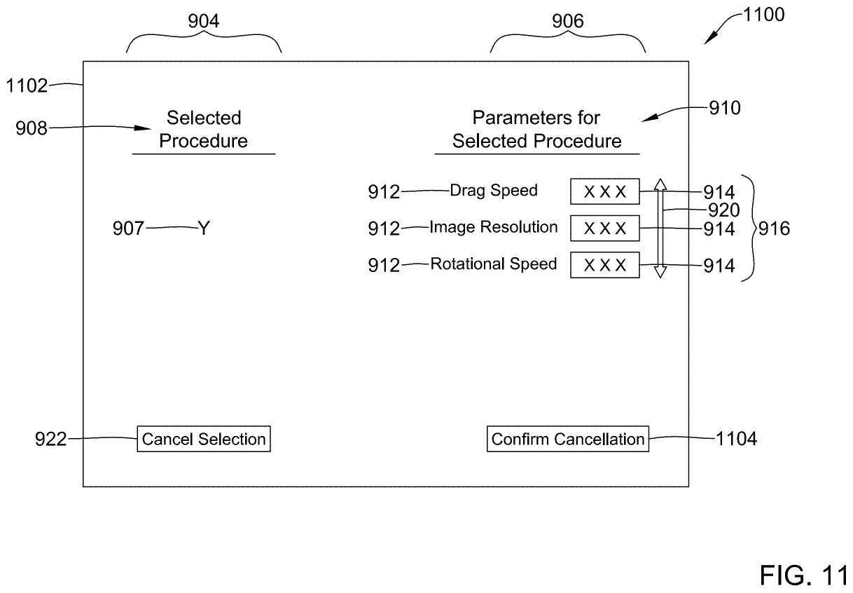

FIG. 11 illustrates another version of the graphical user interface of FIG. 9 that has been interacted with using voice activated commands. FIG. 11 is an illustration of a graphical user interface 1100 including at least a portion of a visual display 1102 that may be provided in response to a voice activated command received by an IVUS imaging system. Embodiments of visual display including visual display 1102 may be provided on display device 304 in response to a voice activated command received at system 300 indicating a request for cancellation of the selected procedure previously being displayed on display device 304, such as the graphical user interface 900 as illustrated and described above with respect to FIG. 9.

Referring to FIG. 11, visual display 1102 includes many of the same elements illustrated and described above with respect to FIG. 9, including headings 908 and 910, a procedure indicator 907 indicating the selected procedure, and a display of a set of parameters and parameter values, as generally indicated by bracket 916. FIG. 11 and visual display 1102 differ from FIG. 9 and visual display 902 in that in FIG. 11, the status box 922 now includes an indication, such as the word “CANCELED,” or similar, along with the addition of a confirm cancellation box 1104. The confirm cancellation box 1104 may include text, such as, “CONFIRM CANCELLATION,” or similar. When displayed, system 300 may be configured to receive a voice activated command, such as, “[Keyword], confirm cancellation of selected procedure,” or similar, that can be used to provide an indication to system 300 to unselect the procedure that was most recently selected.

In some embodiments, upon receiving the voice activated command indicating confirmation of the cancellation of the selected procedure, the display being provided by display device 304 may return to a different display, for example to the visual display 802 as illustrated and described with respect to FIG. 8, which displays a listing of available pre-programmed procedures that may be used to operate the IVUS imaging system.

FIGS. 12A-12B illustrate various stages of a graphical user interfaces 1200 being interacted with using voice activated commands to create a new imaging procedure. FIGS. 12A-12B are illustrations of graphical user interface 1200 including at least a portion of a visual display 1202 that may be provided in response to a voice activated command(s) received by an IVUS imaging system. As shown in FIG. 12A, visual display 1202 includes a first column 1204 of information including a first header 1208 labeled “New Procedure,” an identifier text box 1207 arranged vertically below the first header 1208, and a status box 1222 arranged vertically below the identifier text box 1207. Visual display 1202 further includes a second column 1206 of information including a second header 1210 reciting “Parameters,” and a listing of parameter as generally indicated by bracket 1216, including titles 1212 and associated parameter value boxes 1214 arranged vertically below the second header 1210.

As shown in FIG. 12A, the titles 1212 listed for parameters include parameter 1, parameter 2, and dotted line extending to parameter X. As such, it is shown that the number of parameters that may be listed under the second header 1210 is not limited to any number of parameters. In various embodiments, the number and types of parameters listed under the second header 1210 may be automatically generated by the system to include the parameters that are known to be required for the operation of the IVUS imaging system. The number, type, and arrangement of the listing of the parameters may be modified using voice activated commands.

In various embodiments, the identifier text box 1207 may be automatically filled in with an identifier, such as a number identification, when the screen including visual display 1202 is first generated. In some embodiments, the identifier text box may be filled in for example using a number, a word or words, or a combination of number(s) and word(s) based on information provided to the IVUS imaging system using voice activated commands. In various embodiments, the status box 1222 may be filled with a message including word(s) or some other type of graphical indicator related to the status of the new procedure, for example “in process” or “completed” or any other status message appropriate to reflect the status of the new procedure.

As shown in FIG. 12A, a display of a graphical symbol, such as a dashed line, is generated and positioned within each of the parameter value boxes 1214. In various embodiments, the dashed line, or some other pre-determined graphical depiction, is used to shown that a parameter value, or a range of values where appropriate, has not yet been received and applied to the corresponding parameter.

In various embodiments, voice activated command(s) may be received by the IVUS imaging system, the voice activated command(s) include command instructions including values and/or ranges of values to be applied to one or more of the parameters listed under the second header 1210. As the values and/or ranges of values are received by the system, a graphic representing the value or range of values may be displayed within the parameter value boxes 1214 associated with the corresponding parameter. FIG. 12B illustrates an embodiment of this process, wherein the parameter value boxes 1214 in FIG. 12B are filled with graphical depictions, represented by the “XXX” in the figure, wherein the graphical depiction illustratively represented by “XXX” show the values associated with the respective parameters have been received by the system.

In various embodiments, if the system determines that a value or a range of values received in a voice activated command is improper or out of bounds of a pre-determined range for the associated parameter, the system can be configured to generate an error message, for example that can be provided in the status box 1222, and/or may cause the visual display 1202 to generate some other type of graphical depiction that is indicative of an error, for example to flash on and off the title of the parameter associated with the receipt of the improper parameter value(s).

As further illustrated in FIG. 12B, when all the parameters included under the second header 1210 have been assigned a proper value or ranges of values, status box 1222 may be configured to display a message, such as “COMPLETE,” or similar, indicating that the parameter values have been received for all the listed parameters. Embodiments of visual display 1202 may include a text box 1224 that appears when the status of the process is indicative as being completed, wherein a voice activated command may then be used to confirm that the displayed setting are in fact completed, and causes the system to store the newly created procedure using the identifier, the listing of the parameters and associated parameters values, as a procedure that is available for selection for operating the IVUS imaging system.

FIG. 13 illustrates an embodiment of a first layout 1300 for a visual display 1302 for an IVUS imaging system that may be interacted with using voice activated commands. As shown in FIG. 13, visual display 1302 includes an area of the display that may be divided into a plurality of pre-defined display areas or regions. As a non-limiting example, pre-defined display areas may include a first menu 1302A positioned vertically along the left-hand side of the visual display, and a second menu 1302B positioned vertically along the right-hand side of the visual display. A cross-sectional view area 1304 may be positioned in the center portion of the visual display 1302, extending between the first menu 1302A and the second menu 1302B. Cross-sectional view area 1304 may be an area of the visual display allocated to displaying images in cross-section captured from a vessel, such as a vessel of a cardiac structure. Embodiments of visual display 1302 may also include a vessel longview area 1310 positioned below the cross-sectional view area 1304, and extending between the first menu 1302A and the second menu 1302B, and/or a vessel navigation area 1306 positioned below the vessel longview area 1310. Embodiments of visual display 1302 may incorporate additional display regions for display of a command list 1314 showing a listing of the command words and phrases the system is trained to recognize, and listing of synonyms 1316 associated with the recognized command words, phrases, and instructions, and/or an assessment region 1312 allocated for the display of assessment data.

In various embodiments, the IVUS imaging system may be configured to receive and operate on voice activated commands configured to change or otherwise manipulate the layout and/or the format of the graphical information being displayed or to be displayed on the visual display 1302. For example, a voice activated command may include a keyword followed by a command instruction to show or to hide various information, such as menus, image views, assessment information, and/or listing that is currently being displayed on visual display 1302 or that a user wants to have displayed on the display.

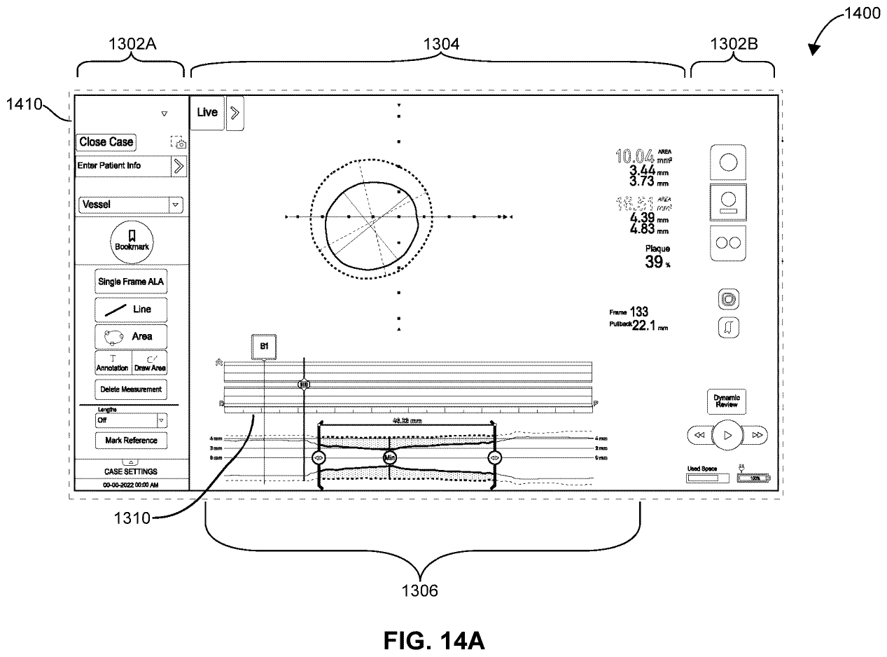

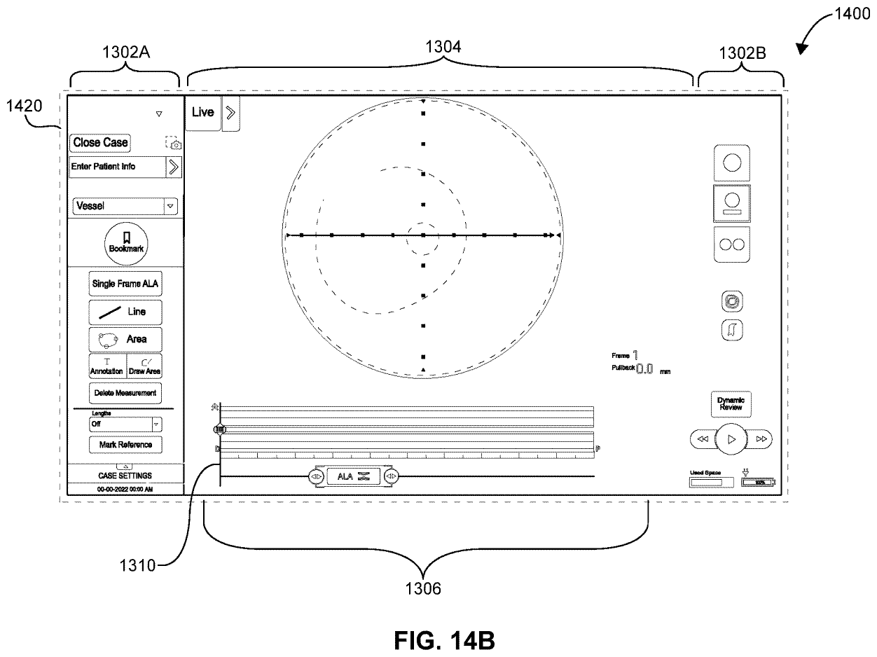

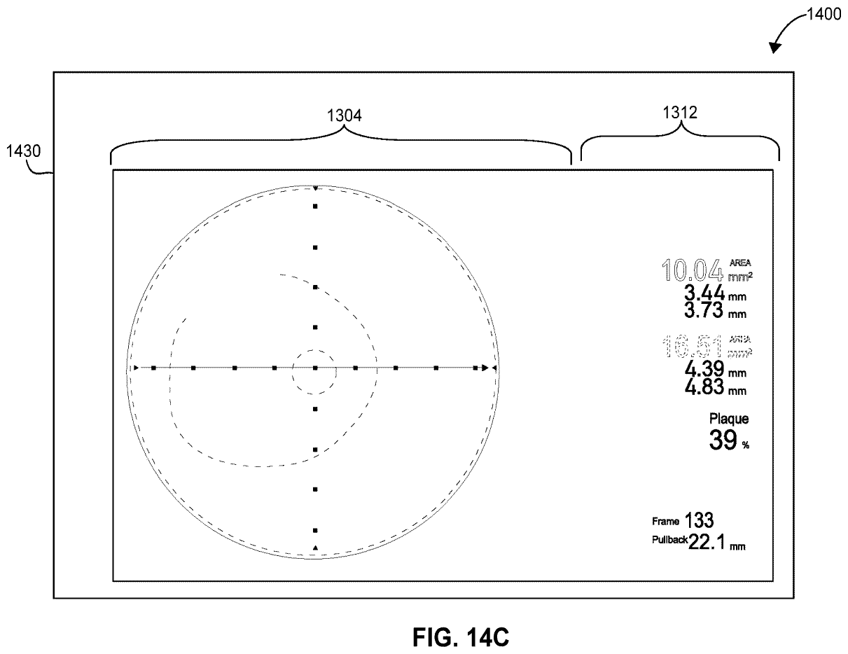

FIGS. 14A, 14B, and 14C illustrate versions of the graphical user interfaces using the first layout of FIG. 13 and that can be or have been interacted with using voice activated commands. FIG. 14A illustrates of a non-limiting example of a graphical user interface 1400 having a visual display 1410 including predefined display areas arranged in a manner illustrated and described above with respect to FIG. 13. As shown in FIG. 14A, visual display 1410 includes first menu 1302A positioned vertically along the left-hand side of the visual display with various text and graphical elements displayed, and second menu 1302B positioned vertically along the right-hand side of the visual display, also with various text and graphical elements displayed. The cross-sectional view area 1304 is positioned in the center portion of the visual display 1302, extending between the first menu 1302A and the second menu 1302B, and includes an image in cross-section of a vessel of a cardiac structure.

Visual display 1410 also includes an image of a vessel in the vessel longview area 1310 positioned below the cross-sectional view area 1304, and extending between the first menu 1302A and the second menu 1302B, and a graphical representation of a navigational tool displayed in the vessel navigation area 1306 positioned below the vessel longview area 1310. For any of these predefined display areas, voice activated commands can be provided to the IVUS imaging system to manipulate the layout and/or content of visual display 1410, for example to hide predefined display area, to enlarge an image, such as a cross-sectional image of the vessel being displayed in the cross-sectional view area 1304, and/or to add other information, such as a listing of the words, phrases, and instructions that the imaging system is configured to recognize as voice activated commands.

FIG. 14B includes visual display 1420 that may be generated and displayed based on voice activated commands received at the IVUS imaging system. For example, starting with a visual display 1410 as illustrated in FIG. 14A, voice activated commands may be received and processed at the IVUS imaging system requesting the cross-sectional view of the imaged vessel being provided as part of the graphical display in cross-sectional view area 1304 be enlarged. The ability to generate the visual display 1420 using voice activated commands can be helpful for allowing a larger image of the cross-sectional view of the imaged vessel to be provided on the display screen, which can be helpful for a user, such as a physician, when reviewing the cross-sectional images.

FIG. 14C includes visual display 1430 that may be generated and displayed based on voice activated commands received at the IVUS imaging system. For example, starting with a visual display 1410 as illustrated in FIG. 14A, voice activated commands may be received and processed at the IVUS imaging system requesting that menus 1302A and 1302B be “hidden,” resulting in removal of these portions of the visual display. In addition, voice activated commands may be received and processed at the IVUS imaging system requesting that the vessel longview area 1310 and the vessel navigation area 1306 also be “hidden,” resulting in removal of the graphical depictions associated with these areas, wherein the graphical elements previously displayed in these predefined viewing areas will no longer be displayed as part of the visual display 1430. The resulting visual display 1430, and as illustrated in FIG. 14C, includes an enlarged cross-sectional view area 1304 and associated data fields related to assessment region 1312 remaining on the visual display. The ability to generate the visual display 1430 using voice activated commands can be helpful for allowing a larger image of the cross-sectional view of the imaged vessel to be provided on the display screen, which can be helpful for a user, such as a physician, when reviewing the cross-sectional images.

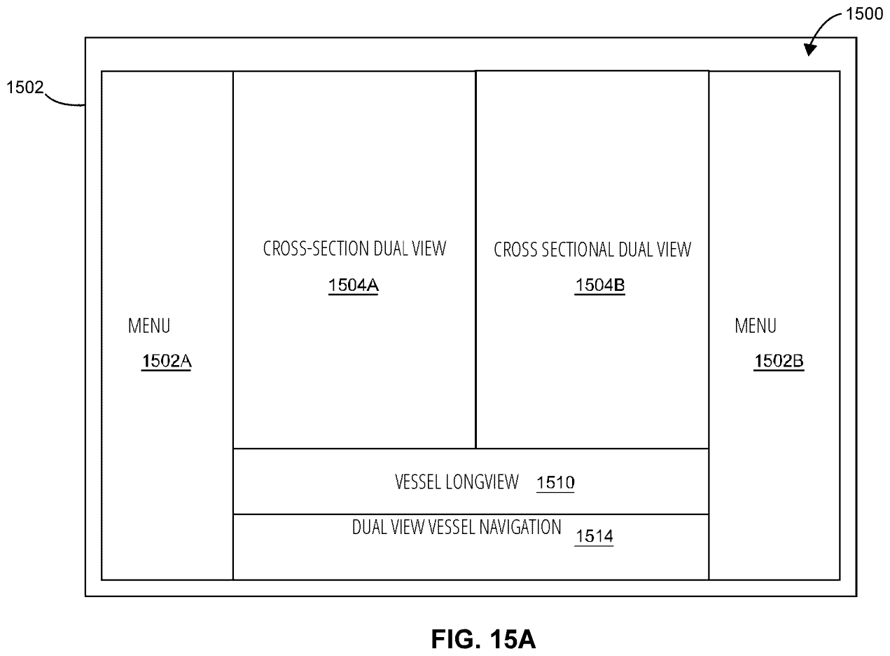

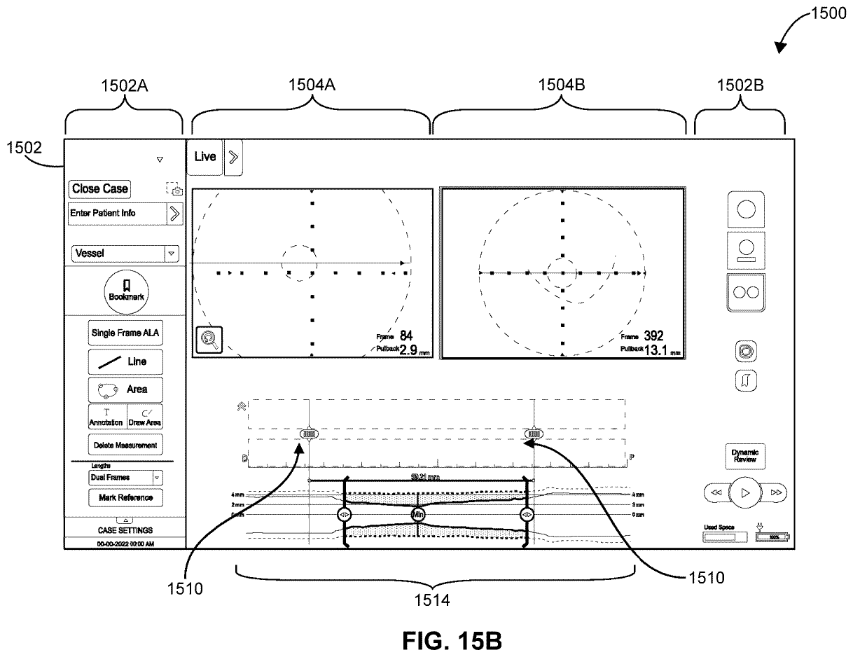

FIG. 15A illustrates an embodiment of a second layout for a graphical display 1500 for an IVUS imaging system that may be interacted with using voice activated commands. FIG. 15A includes an illustration of at least a portion of a visual display 1502 that may be provided in response to a voice activated commands received by an IVUS imaging system. Some of the predefined display area as shown in FIG. 15A for visual display 1502 correspond to the same or similar predefined display regions illustrated and described above with respect to FIG. 13, including first menu 1502A positioned vertically along the left-hand side of the visual display, and a second menu 1502B positioned vertically along the right-hand side of the visual display. Visual display 1502 also includes an image of a vessel in a vessel longview area 1510 positioned below the dual cross-sectional view areas 1504A, 1504B, and extending between the first menu 1502A and the second menu 1502B, and a graphical representation of a navigational tool displayed in a vessel navigation area 1514 positioned below the vessel longview area 1510.

The visual display 1502 in FIG. 15A differs from the visual display 1302 in FIG. 13 in that the cross-sectional viewing area in the center portion of visual display 1502 is split into two separate cross-sectional views 1504A and 1504B. This feature of visual display 1502 can be useful to a user, such as a physician, to allow comparisons on a same display between different potions of an imaged vessel, or for example comparison of cross-sectional views of a same portion of a vessel but imaged at different times. In various embodiments, the split views provided in the cross-sectional viewing area of visual display 1502 may be rendered on user graphical display 1500 in response to one or more voice activated command received and processed at the IVUS imaging system.

FIG. 15B illustrates a version of graphical user interfaces 1500 using the second layout of FIG. 15A and that can be or has been interacted with using voice activated commands. As shown in FIG. 15B, visual display 1502 includes various data values and graphical symbols displayed as part of menus 1502A and 1502B, a set of dual cross-sectional images 1504A and 1504B provide in the cross-sectional view area, and graphical representations included in vessel longview area 1510 and in the dual view vessel navigation area 1514. The general arrangement and location within the visual display 1502 of the menus, cross-sectional views, along with vessel longviews and navigational tools, may be based on the allocated display areas as defined in FIG. 15A.

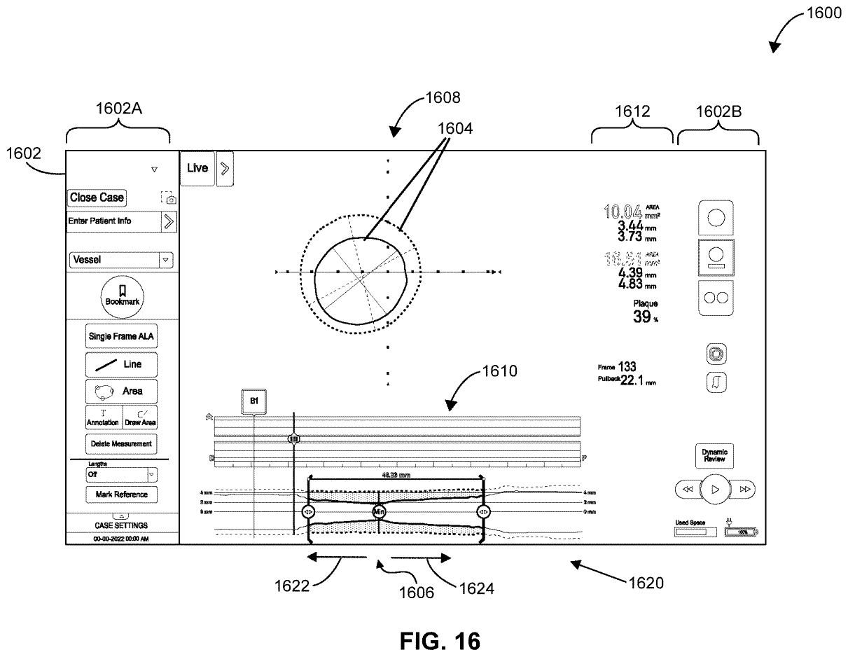

FIG. 16 illustrates a graphical user interface 1600 showing a navigational bar 1620 that can be interacted with using voice activated commands. FIG. 16 illustrates at least a portion of a visual display 1602 that may be provided in response to a voice activated command received by an IVUS imaging system. The general arrangement and location within the display 1602 of the menus, cross-sectional area views, and other display areas may be based on the allocated display areas as defined in FIG. 13. As shown in FIG. 16, visual display 1602 including menus 1602A and 1602B, along with a cross-sectional view area including cross-sectional image 1608 and along with associated data 1612, vessel longview area 1610, and vessel navigation area, which in various embodiments correspond to the display areas having a general layout and arrangement as illustrated and described above with respect to FIG. 13.

Referring to FIG. 16, the vessel navigation area includes vessel longview 1610 that includes a graphical depiction of the images captured along the length of a vessel imaged using the IVUS imaging system. Navigation bar 1620 includes a slider bar 1606 that provides a visual indication of which portion along the imaged vessel is being depicted by the cross-sectional view provided in the cross-sectional view area 1604. By moving the slider bar 1606 to the left or right, as illustratively represented by arrows 1622 and 1624, respectively, the portion of the vessel that is being depicted by the cross-sectional view being provided in the cross-sectional view area 1604 may be moved. In various embodiments, voice activated commands can be received and processed at the IVUS imaging system that include command instructions to move the slider bar 1606 to the left or to the right, thereby allowing the voice activated commands to control which portion of the imaged vessels is being displayed in the cross-sectional view area 1604. This ability to alter the portion of imaged vessel being displayed in the cross-sectional view area 1604 using voice activated commands can be helpful to a user, such as a physician, in performing an analysis and review of the status and condition of the imaged portion of the vessel.

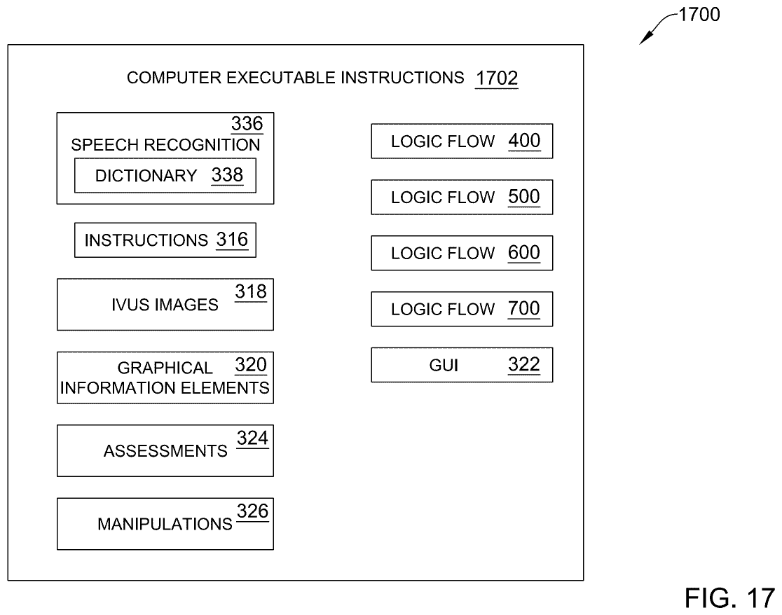

FIG. 17 illustrates computer-readable storage medium 1700. Computer-readable storage medium 1700 may comprise any non-transitory computer-readable storage medium or machine-readable storage medium, such as an optical, magnetic or semiconductor storage medium. In various embodiments, computer-readable storage medium 1700 may comprise an article of manufacture. In some embodiments, computer-readable storage medium 1700 may store computer executable instructions 316 with which circuitry (e.g., processor 106, processor 306, IVUS imaging system acquisition circuitry 314, and the like) can execute. For example, computer executable instructions 1702 can include instructions to implement operations described with respect to instructions 316, IVUS images 318, graphical information elements 320, assessments 324, manipulations 326, speech recognition software 336, and/or GUI 322, as well as logic flows 400, 500, 600, and/or 700. Examples of computer-readable storage medium 1700 or machine-readable storage medium may include any tangible media capable of storing electronic data, including volatile memory or non-volatile memory, removable or non-removable memory, erasable or non-erasable memory, writeable or re-writeable memory, and so forth. Examples of computer executable instructions 1702 may include any suitable type of code, such as source code, compiled code, interpreted code, executable code, static code, dynamic code, object-oriented code, visual code, and the like.

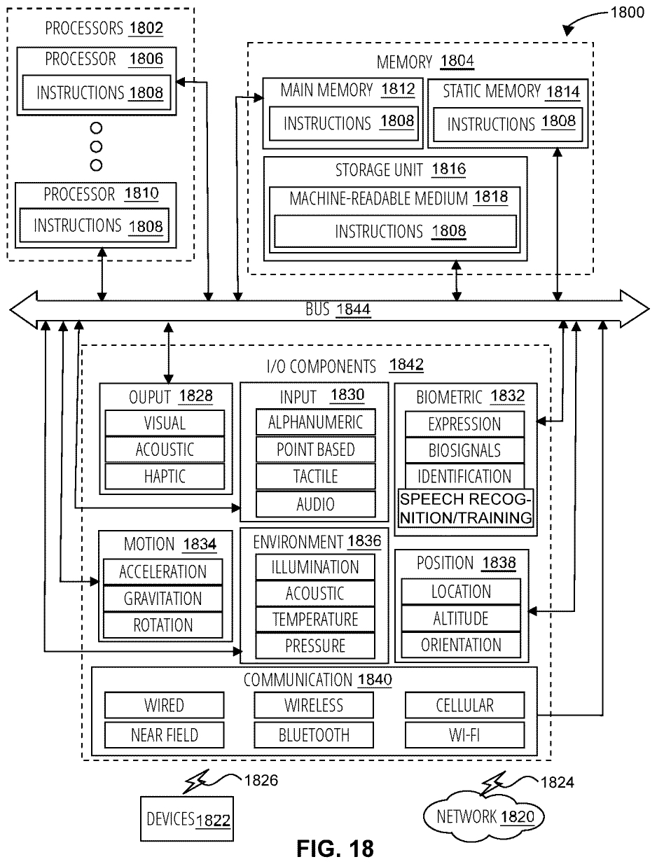

FIG. 18 illustrates a diagrammatic representation of a machine 1800 in the form of a computer system within which a set of instructions may be executed for causing the machine to perform any one or more of the methodologies discussed herein. More specifically, FIG. 18 shows a diagrammatic representation of the machine 1800 in the example form of a computer system, within which instructions 1808 (e.g., software, a program, an application, an applet, an app, or other executable code) for causing the machine 1800 to perform any one or more of the methodologies discussed herein may be executed. For example, the instructions 1808 may cause the machine 1800 to execute instructions 316 of FIG. 3, logic flow 400 of FIG. 4, logic flow 500 of FIG. 5, logic flow 600 of FIG. 6 and logic flow 700 of FIG. 7, and any of the speech recognition functions as described here, and any equivalents thereof. More generally, the instructions 1808 may cause the machine 1800 to receive and process audio inputs comprising voice activated commands, and to generate outputs, including instructions to control imaging procedures using the IVUS imaging system and/or to control the generation of GUIs with functionality and behavior as described herein during a pre-PCI, peri-PCI, or post-PCI based on the received voice activated commands. It is noted that the present disclosure provides specific and discrete implementations of the use of voice activated commands to control the operation of imaging procedures and to control and configure GUI representations and behavior that is a significant improvement over the prior art. In particular, the present disclosure provides an improvement to computing technology in the ability to receive and process voice activated commands associated with the operation of IVUS imaging system and control over the type and arrangement of graphical information provided on a display device associated with the imaging system in order to provide for better throughput and the ability to maintain a sterile environment in the area utilizing the imaging system to perform various medical procedures, including but not limited to IVUS imaging procedures.

The instructions 1808 transform the general, non-programmed machine 1800 into a particular machine 1800 programmed to carry out the described and illustrated functions in a specific manner. In alternative embodiments, the machine 1800 operates as a standalone device or may be coupled (e.g., networked) to other machines. In a networked deployment, the machine 1800 may operate in the capacity of a server machine or a client machine in a server-client network environment, or as a peer machine in a peer-to-peer (or distributed) network environment. The machine 1800 may comprise, but not be limited to, a server computer, a client computer, a personal computer (PC), a tablet computer, a laptop computer, a netbook, a set-top box (STB), a PDA, an entertainment media system, a cellular telephone, a smart phone, a mobile device, a wearable device (e.g., a smart watch), a smart home device (e.g., a smart appliance), other smart devices, a web appliance, a network router, a network switch, a network bridge, or any machine capable of executing the instructions 1808, sequentially or otherwise, that specify actions to be taken by the machine 1800. Further, while only a single machine 1800 is illustrated, the term “machine” shall also be taken to include a collection of machines 1800 that individually or jointly execute the instructions 1808 to perform any one or more of the methodologies discussed herein.

The machine 1800 may include processors 1802, memory 1804, and I/O components 1842, which may be configured to communicate with each other such as via a bus 1844. In an example embodiment, the processors 1802 (e.g., a Central Processing Unit (CPU), a Reduced Instruction Set Computing (RISC) processor, a Complex Instruction Set Computing (CISC) processor, a Graphics Processing Unit (GPU), a Digital Signal Processor (DSP), an ASIC, a Radio-Frequency Integrated Circuit (RFIC), another processor, or any suitable combination thereof) may include, for example, a processor 1806 and a processor 1810 that may execute the instructions 1808. The term “processor” is intended to include multi-core processors that may comprise two or more independent processors (sometimes referred to as “cores”) that may execute instructions contemporaneously. Although FIG. 18 shows multiple processors 1802, the machine 1800 may include a single processor with a single core, a single processor with multiple cores (e.g., a multi-core processor), multiple processors with a single core, multiple processors with multiples cores, or any combination thereof.

The memory 1804 may include a main memory 1812, a static memory 1814, and a storage unit 1816, both accessible to the processors 1802 such as via the bus 1844. The main memory 1804, the static memory 1814, and storage unit 1816 store the instructions 1808 embodying any one or more of the methodologies or functions described herein. The instructions 1808 may also reside, completely or partially, within the main memory 1812, within the static memory 1814, within machine-readable medium 1818 within the storage unit 1816, within at least one of the processors 1802 (e.g., within the processor's cache memory), or any suitable combination thereof, during execution thereof by the machine 1800.

The I/O components 1842 may include a wide variety of components to receive input, provide output, produce output, transmit information, exchange information, capture measurements, and so on. The specific I/O components 1842 that are included in a particular machine will depend on the type of machine. For example, portable machines such as mobile phones will likely include a touch input device or other such input mechanisms, while a headless server machine will likely not include such a touch input device. It will be appreciated that the I/O components 1842 may include many other components that are not shown in FIG. 18. The I/O components 1842 are grouped according to functionality merely for simplifying the following discussion and the grouping is in no way limiting. In various example embodiments, the I/O components 1842 may include output components 1828 and input components 1830. The output components 1828 may include visual components (e.g., a display such as a plasma display panel (PDP), a light emitting diode (LED) display, a liquid crystal display (LCD), a projector, or a cathode ray tube (CRT)), acoustic components (e.g., speakers), haptic components (e.g., a vibratory motor, resistance mechanisms), other signal generators, and so forth. The input components 1830 may include alphanumeric input components (e.g., a keyboard, a touch screen configured to receive alphanumeric input, a photo-optical keyboard, or other alphanumeric input components), point-based input components (e.g., a mouse, a touchpad, a trackball, a joystick, a motion sensor, or another pointing instrument), tactile input components (e.g., a physical button, a touch screen that provides location and/or force of touches or touch gestures, or other tactile input components), audio input components (e.g., a microphone), and the like.

In further example embodiments, the I/O components 1842 may include biometric components 1832, motion components 1834, environmental components 1836, or position components 1838, among a wide array of other components. For example, the biometric components 1832 may include components to detect expressions (e.g., hand expressions, facial expressions, vocal expressions, body gestures, or eye tracking), measure biosignals (e.g., blood pressure, heart rate, body temperature, perspiration, or brain waves), identify a person (e.g., voice identification, retinal identification, facial identification, fingerprint identification, or electroencephalogram-based identification), and the like. Biometric components 1832 also include speech recognition and training components, such as speech recognition software configured to perform any of the speech recognition functions described herein with respect to use of voice activated commands, and with respect to training for example to recognize a user's acescents and personal voice inflections. The motion components 1834 may include acceleration sensor components (e.g., accelerometer), gravitation sensor components, rotation sensor components (e.g., gyroscope), and so forth. The environmental components 1836 may include, for example, illumination sensor components (e.g., photometer), temperature sensor components (e.g., one or more thermometers that detect ambient temperature), humidity sensor components, pressure sensor components (e.g., barometer), acoustic sensor components (e.g., one or more microphones that detect background noise), proximity sensor components (e.g., infrared sensors that detect nearby objects), gas sensors (e.g., gas detection sensors to detection concentrations of hazardous gases for safety or to measure pollutants in the atmosphere), or other components that may provide indications, measurements, or signals corresponding to a surrounding physical environment. The position components 1838 may include location sensor components (e.g., a GPS receiver component), altitude sensor components (e.g., altimeters or barometers that detect air pressure from which altitude may be derived), orientation sensor components (e.g., magnetometers), and the like.

Communication may be implemented using a wide variety of technologies. The I/O components 1842 may include communication components 1840 operable to couple the machine 1800 to a network 1820 or devices 1822 via a coupling 1824 and a coupling 1826, respectively. The I/O devices 1822 may include one or more input and/or output devices configured to provide inputs and/or output, respectively, to the system for use in conjunction with operating the system using voice activated commands. For example, I/O devices 1822 may include audio input devices 330 (FIG. 3), which may include one or more individual microphones and/or one or more microphones provided as part of headset(s), and configured to allow for audio sounds, such as voices, to be detected and input into the system for the purpose of controlling the system using voice activated commands. Further, I/O devices 1822 may include audio output devices 332 (FIG. 3), which may include one or more individual audio output devices such as speaker and/or headphones provided as part of headset(s), and configured to allow for audio sounds, such as computer-generated speech, to be output as audio sound for a user to hear, and may be provided as part of a set of computer-generated audio responses issued as part of operating the system using voice activated commands.

In various embodiments, the communication components 1840 may include a network interface component or another suitable device to interface with the network 1820. In further examples, the communication components 1840 may include wired communication components, wireless communication components, cellular communication components, Near Field Communication (NFC) components, Bluetooth® components (e.g., Bluetooth® Low Energy), Wi-Fi® components, and other communication components to provide communication via other modalities. The devices 1822 may be another machine or any of a wide variety of peripheral devices (e.g., a peripheral device coupled via a USB).

Moreover, the communication components 1840 may detect identifiers or include components operable to detect identifiers. For example, the communication components 1840 may include Radio Frequency Identification (RFID) tag reader components, NFC smart tag detection components, optical reader components (e.g., an optical sensor to detect one-dimensional bar codes such as Universal Product Code (UPC) bar code, multi-dimensional bar codes such as Quick Response (QR) code, Aztec code, Data Matrix, Dataglyph, MaxiCode, PDF417, Ultra Code, UCC RSS-2D bar code, and other optical codes), or acoustic detection components (e.g., microphones to identify tagged audio signals). In addition, a variety of information may be derived via the communication components 1840, such as location via Internet Protocol (IP) geolocation, location via Wi-Fi® signal triangulation, location via detecting an NFC beacon signal that may indicate a particular location, and so forth.

The various memories (i.e., memory 1804, main memory 1812, static memory 1814, and/or memory of the processors 1802) and/or storage unit 1816 may store one or more sets of instructions and data structures (e.g., software) embodying or utilized by any one or more of the methodologies or functions described herein. These instructions (e.g., the instructions 1808), when executed by processors 1802, cause various operations to implement the disclosed embodiments.

As used herein, the terms “machine-storage medium,” “device-storage medium,” “computer-storage medium” mean the same thing and may be used interchangeably in this disclosure. The terms refer to a single or multiple storage devices and/or media (e.g., a centralized or distributed database, and/or associated caches and servers) that store executable instructions and/or data. The terms shall accordingly be taken to include, but not be limited to, solid-state memories, and optical and magnetic media, including memory internal or external to processors. Specific examples of machine-storage media, computer-storage media and/or device-storage media include non-volatile memory, including by way of example semiconductor memory devices, e.g., erasable programmable read-only memory (EPROM), electrically erasable programmable read-only memory (EEPROM), FPGA, and flash memory devices; magnetic disks such as internal hard disks and removable disks; magneto-optical disks; and CD-ROM and DVD-ROM disks. The terms “machine-storage media,” “computer-storage media,” and “device-storage media” specifically exclude carrier waves, modulated data signals, and other such media, at least some of which are covered under the term “signal medium” discussed below.

In various example embodiments, one or more portions of the network 1820 may be an ad hoc network, an intranet, an extranet, a VPN, a LAN, a WLAN, a WAN, a WWAN, a MAN, the Internet, a portion of the Internet, a portion of the PSTN, a plain old telephone service (POTS) network, a cellular telephone network, a wireless network, a Wi-Fi® network, another type of network, or a combination of two or more such networks. For example, the network 1820 or a portion of the network 1820 may include a wireless or cellular network, and the coupling 1824 may be a Code Division Multiple Access (CDMA) connection, a Global System for Mobile communications (GSM) connection, or another type of cellular or wireless coupling. In this example, the coupling 1824 may implement any of a variety of types of data transfer technology, such as Single Carrier Radio Transmission Technology (1xRTT), Evolution-Data Optimized (EVDO) technology, General Packet Radio Service (GPRS) technology, Enhanced Data rates for GSM Evolution (EDGE) technology, third Generation Partnership Project (3GPP) including 3G, fourth generation wireless (4G) networks, Universal Mobile Telecommunications System (UMTS), High Speed Packet Access (HSPA), Worldwide Interoperability for Microwave Access (WiMAX), Long Term Evolution (LTE) standard, others defined by various standard-setting organizations, other long range protocols, or other data transfer technology.

The instructions 1808 may be transmitted or received over the network 1820 using a transmission medium via a network interface device (e.g., a network interface component included in the communication components 1840) and utilizing any one of several well-known transfer protocols (e.g., hypertext transfer protocol (HTTP)). Similarly, the instructions 1808 may be transmitted or received using a transmission medium via the coupling 1826 (e.g., a peer-to-peer coupling) to the devices 1822. The terms “transmission medium” and “signal medium” mean the same thing and may be used interchangeably in this disclosure. The terms “transmission medium” and “signal medium” shall be taken to include any intangible medium that can store, encoding, or carrying the instructions 1808 for execution by the machine 1800, and includes digital or analog communications signals or other intangible media to facilitate communication of such software. Hence, the terms “transmission medium” and “signal medium” shall be taken to include any form of modulated data signal, carrier wave, and so forth. The term “modulated data signal” means a signal that has one or more of its characteristics set or changed in such a matter as to encode information in the signal.

Terms used herein should be accorded their ordinary meaning in the relevant arts, or the meaning indicated by their use in context, but if an express definition is provided, that meaning controls.

Herein, references to “one embodiment” or “an embodiment” do not necessarily refer to the same embodiment, although they may. Unless the context clearly requires otherwise, throughout the description and the claims, the words “comprise,” “comprising,” and the like are to be construed in an inclusive sense as opposed to an exclusive or exhaustive sense; that is to say, in the sense of “including, but not limited to.” Words using the singular or plural number also include the plural or singular number respectively, unless expressly limited to one or multiple ones. Additionally, the words “herein,” “above,” “below” and words of similar import, when used in this application, refer to this application as a whole and not to any portions of this application. When the claims use the word “or” in reference to a list of two or more items, that word covers all the following interpretations of the word: any of the items in the list, all the items in the list and any combination of the items in the list, unless expressly limited to one or the other. Any terms not expressly defined herein have their conventional meaning as commonly understood by those having skill in the relevant art(s).

What is claimed is:

1. An intravascular ultrasound (IVUS) imaging system, comprising:
an audio input device;
a display device;
an IVUS catheter;
a computing device including a processor coupled to the audio input device, the display device, and the IVUS catheter; and
a memory device comprising instructions, which when executed by the processor cause the IVUS imaging system to:
receive from the audio input device an audio signal representative of an audio sound input;
process the audio signal to detect an indication of a voice activated command included in the audio sound input; and
upon detection of the indication of the voice activated command, configure one or more operating parameters of the IVUS imaging system based on a command instruction included in the voice activated command.

2. The IVUS imaging system of claim 1, wherein the voice activated command comprises a keyword followed by the command instruction.

3. The IVUS imaging system of claim 1, the memory storage device further comprising instructions executable by the processor, which instructions when executed cause the IVUS system to:
process the audio signal using speech recognition software to detect the indication of a voice activated command included in the audio sound input.

4. The IVUS imaging system of claim 3, the memory storage device further comprising instructions executable by the processor, which instructions when executed cause the IVUS system to:
create a voice training file using machine learning to train the speech recognition software to detect and remove an accent from the audio sound input.

5. The IVUS imaging system of claim 3, the memory storage device further comprising instructions executable by the processor, which instructions when executed cause the IVUS system to:
process the audio signal using the speech recognition software includes the use of an algorithm comprising steps configured to:
monitor the audio signal representative of audio sound input detected at the audio input device;
detect the presence of a keyword in the audio signal;
process a subsequent portion of the audio signal to determine that the input signal includes representations of recognized command words or instructions; and
generate and output one or more control commands, the one or more control commands configured to control one or more aspects of the operation of one or more devices included in the IVUS imaging system.

6. The IVUS imaging system of 5, the memory storage device further comprising instructions executable by the processor, which instructions when executed cause the IVUS system to:
control the operation of an imaging catheter while the imaging catheter is operated to perform an imaging procedure while positioned in a vessel of a patient.

7. The IVUS imaging system of 5, wherein the one or more control commands are configured to control a content and a format of a visual display being provided on a display device coupled with the IVUS imaging system.

8. The IVUS imaging system of claim 1, wherein the one or more operating parameters of the IVUS imaging system based on the command instruction included in the voice activated command comprise instructions executable by the processor, which instructions when executed cause the IVUS system to:
determine that the command instruction includes a request to display of list of procedures available for performing an imaging procedure using the IVUS imaging system;
generate graphical indications comprising the list of procedures that are available for performing the imagines procedure using the IVUS imaging system, and
display, on a display device, the list of procedure.

9. The IVUS imaging system of claim 1, wherein the one or more operating parameters of the IVUS imaging system based on a command instruction included in the voice activated command comprise instructions executable by the processor, which instructions when executed cause the IVUS system to:
determine that the command instruction includes an indication of a selected procedure that was chosen from a list of pre-programmed procedures available for controlling an imaging procedure using the IVUS imaging system;
generate graphical indications comprising a process identifier and a listing of a set of parameters associated with the selected procedure, and
display, on a display device, the process identifier and the set of parameters associated with the selected procedure.

10. The IVUS imaging system of claim 1, the memory storage device further comprising instructions executable by the processor, which instructions when executed cause the IVUS system to:
configure the one or more operating parameters of the IVUS imaging system based on a command instruction comprising instructions to:
determine that the command instruction includes a request to create a new pre-programmed procedure available for controlling an imaging procedure using the IVUS imaging system;
generate graphical indications comprising an interactive listing of a set of parameters required for creating the new pre-programmed procedure;
display, on a display device, the interactive listing of the set of parameters required for creating the new pre-programmed procedure, wherein the interactive listing of the set of parameters is initially displayed without parameter values associated with any of the parameters of the set of parameters; and
assign a parameter value to each of the parameters included in the set of parameters using one or more voice activated commands.

11. The IVUS imaging system of claim 1, wherein the audio input device comprises a microphone incorporated into a headset.

12. The IVUS imaging system of claim 1, the memory storage device further comprising instructions executable by the processor, which instructions when executed cause the IVUS system to:
perform an imaging procedure using the IVUS imaging system, the imaging procedure performed based at least in part on the one or more operating parameters configured based on the command instruction included in the voice activated command.

13. The IVUS imaging system of claim 12, wherein the instructions executable by the processor, which instructions when executed cause the IVUS system to perform the imaging procedure include instructions to:
perform the imaging procedure using an automatic pullback of the imaging catheter through a vessel of a patient, and wherein at least one of the one or more operating parameters including a value for a drag speed for the automatic pullback.

14. At least one machine readable storage device, comprising a plurality of instructions that in response to being executed by a processor of an intravascular ultrasound (IVUS) imaging system cause the processor to:
receive from the audio input device an audio signal representative of an audio sound input;
process the audio signal to detect an indication of a voice activated command included in the audio sound input; and
upon detection of the indication of the voice activated command, configure one or more operating parameters of the IVUS imaging system based on a command instruction included in the voice activated command.

15. The at least one machine readable storage device of claim 14, wherein the voice activated command comprises a keyword followed by the command instruction.

16. The at least one machine readable storage device of claim 14, further comprising instructions that in response to being executed by the processor cause the processor to:
process the audio signal using speech recognition software to detect the indication of a voice activated command included in the audio sound input.

17. The at least one machine readable storage device of claim 14, further comprising instructions that in response to being executed by the processor cause the processor to:
perform an imaging procedure using the IVUS imaging system, the imaging procedure performed based at least in part on the one or more operating parameters configured based on the command instruction included in the voice activated command.

18. A method for an intravascular ultrasound (IVUS) imaging system, comprising:
receiving, from an audio input device coupled to a computing device, an audio signal representative of an audio sound input;
processing the audio signal to detect an indication of a voice activated command included in the audio sound input; and
upon detection of the indication of the voice activated command, configuring one or more operating parameters of the IVUS imaging system based on a command instruction included in the voice activated command.

19. The method of claim 18, wherein the voice activated command comprises a keyword followed by the command instruction.

20. The method of claim 18, further comprising
performing an imaging procedure using the IVUS imaging system, the imaging procedure performed based at least in part on the one or more operating parameters configured based on the command instruction included in the voice activated command.
